(19) United States
(12) Patent Application Publication
(10) Pub. No.: US 2016/0348052 A1
(43) Pub. Date: Dec. 1, 2016

(54) In Vitro Pharmacokinetics/Pharmacodynamics Bellows Perfusion System for Enhancing Effectiveness of Cancer Chemotherapy

(71) Applicants: HUNG-YUN LIN, SCHENECTADY, NY (US); GEORGE DRUSANO, Windmere, FL (US); ARNOLD LOUIE, ORLANDO, FL (US); PAUL J. DAVIS, WEST SAND LAKE, NY (US)

(72) Inventors: HUNG-YUN LIN, SCHENECTADY, NY (US); GEORGE DRUSANO, Windmere, FL (US); ARNOLD LOUIE, ORLANDO, FL (US); PAUL J. DAVIS, WEST SAND LAKE, NY (US)

(21) Appl. No.: 14/903,149

(22) PCT Filed: Jul. 13, 2011
(86) PCT No.: PCT/US2011/043837

§ 371 Date: Aug. 8, 2016

Related U.S. Application Data
(63) Continuation-in-part of application No. 12/751,375, filed on Mar. 31, 2010, now Pat. No. 9,180,107.
(60) Provisional application No. 61/363,831, filed on Jul. 13, 2010.
(60) Provisional application No. 61/165,119, filed on Mar. 31, 2009.

(51) Int. Cl. C12M 1/12; C12Q 1/68; G01N 33/50; C12M 1/24; C12N 5/09

(52) U.S. Cl. CPC C12M 25/16; C12M 25/10; C12M 23/08; C12N 5/0693; G01N 33/5011; C12Q 1/6886; C12N 2531/00; C12Q 2600/136; C12Q 2600/158

(57) ABSTRACT

Provided herein is a continuous cell perfusion model system that provides useful pharmacokinetic and pharmacodynamic information on the application of new drugs or combinations of various agents in vitro to human cancer cell lines. Also provided are methods of using this system to individualize cancer treatment.

### RELATED APPLICATIONS

This application claims priority to U.S. Ser. No. 61/363,831, filed Jul. 13, 2010 and is a continuation-in-part of U.S. Ser. No. 12/751,375, filed Mar. 31, 2010, which claims priority to U.S. Ser. No. 61/165,119, filed Mar. 31, 2009. Each of these applications is herein incorporated by reference in its entirety.

### FIELD OF THE INVENTION

The invention described herein pertains generally to in vitro cell culture systems methods of using such systems to enhance the effectiveness of cancer treatments.

### BACKGROUND

Clinical treatment protocols for specific solid cancers have favorable response rates of 20%-25%. However, by their nature, such protocols do not individualize or “personalize” cancer treatments, and treatment dosages are standardized on a per kilogram body weight basis, not on any patient tumor-specific dose susceptibility. When solid cancers that initially responded to therapy relapse, these tumors have usually acquired traits that render them resistant to the initial treatment agent(s). Finally, it is also impractical to conduct enough clinical trials to evaluate specific combinations of cancer therapeutic agents in several dosages that might permit optimization of treatment in either the cancer patient who is presenting initially or the cancer patient who is relapsing.

Thus, a need exists for a means to supply a data-driven, personalized cancer chemotherapy regime.

### SUMMARY OF THE INVENTION

Provided herein is an in vitro cell culture system containing (a) a compressible vessel comprising cell culture medium, a plurality of polymer flakes, and one or more porous membranes, wherein the polymer flakes have one or more cancer cells adhered thereto; (b) a media vessel used to supply the cell culture medium to the compressible vessel, wherein the media vessel is attached to the compressible vessel via a first connection line having at least one access point where one or more anti-cancer drugs or chemotherapeutic agents can be added to the cell culture medium; (c) a waste vessel used to remove waste products from the compressible vessel, wherein the waste vessel is attached to the compressible vessel via a second connection line having at least one access point where samples of the cell culture medium from the compressible vessel can be removed for analysis; and (d) a hollow fiber tube that connects the media vessel and the compressible vessel.

In various embodiments, the hollow fiber tube in the in vitro cell culture system of the invention provides an environment for endothelial cells to grow. The hollow fiber tube allows the study of the effect of anti-cancer drug or chemotherapeutic agent-induced anti-angiogenesis. Additionally (or alternatively), the hollow fiber tube allows the study of the effect of angiogenesis induced by cancer cells grown in the compressible vessel in the control set and the study of anti-cancer drug or chemotherapeutic agent-induced anti-angiogenesis. The cell culture medium present in the compressible vessel can be changed over a 24 hour period.

Those skilled in the art will recognize that the compressible vessel of the in vitro cell culture system can be a compressible (bellows) bottle. Moreover, in this in vitro cell culture system the one or more cancer cells spontaneously adhere to the polymer flakes in the compressible vessel and proliferate. In some embodiments, the polymer flakes are treated to enable seeding and the harvesting of cells and secreted proteins. By way of non-limiting example, between 10-15×10⁶ and 30-50×10⁶ cells are attached to the polymer flakes.

When the compressible vessel is compressed, the level of cell culture medium within the compressible vessel changes. By way of non-limiting example, when the compressible vessel is compressed, the cells adhered to the polymer flakes are alternatively submerged in the cell culture medium and exposed to 5% CO₂/95% air. In such an embodiment, there is a dynamic interface between air and medium on the cells adhered to the polymer flakes that maximizes nutrient uptake and oxygen transfer by the cells.

The in vitro cell culture system described herein provides a low shear, high aeration, and foam-free cell culture environment.

Also provided herein are methods of using the in vitro cell culture systems of the invention by seeding 5×10⁷ cell onto polymer flakes in perfusion and non-perfusion bellows bottles; incubating the cells overnight; harvesting the flakes; placing the harvested flakes in the compressible vessel and allowing the system to run for at least 2 days; exposing the cultured cells to serum-containing medium; adding an amount of one or more anti-cancer drugs or chemotherapeutic agents via the access point in the first connection line for a period of up to 60 days (e.g., 1 to 21 days). In some embodiments, the compressible vessel is alternatively compressed and released during the period of up to 60 days (e.g., 1 to 21 days). In one preferred embodiment, the cells are incubated overnight at 37° C. However, those skilled in the art will recognize that the incubation temperature can be adjusted higher or lower to match the clinical treatment protocols of certain cancer patients (i.e., hyperthermia or hypothermia).

In the methods of the invention, the amount of the one or more anti-cancer drugs or chemotherapeutic agents is constant. Additionally (or alternatively), the one or more anti-cancer drugs or chemotherapeutic agents is added by infusion and/or periodically. Moreover, the amount of the one or more anti-cancer drugs or chemotherapeutic agents can be varied by adjusting the flow rather of the cell culture medium, the dosing schedule, or both. For example, the one or more anti-cancer drugs or chemotherapeutic agents can be administered in multiple short-term infusions, in intermittent infusions, in brief injections, or in any combination thereof.

Any of the methods disclosed herein can also include the step of harvesting one or more of: cell culture medium; soluble metabolites for cell culture; metabolites or anti-cancer drugs or chemotherapeutic agents; or any combination thereof from the access point in the second connection line for analysis. For example, such analysis can include, but is not limited to, one or more pharmacodynamic studies selected from flow cytometry for cell cycle arrest or subpopulations of cells; microarray analysis for gene profiles; RT-PCR for gene expression; Western blot for anti-apoptotic or pro-apoptotic proteins; and/or direct cell counts.

Additionally, the methods of the invention can also include the step of harvesting cells from the polymer flakes for analysis. By way of non-limiting example, the analysis of the harvested cells can be selected from measurements of cell cycling, measurement of apoptosis, analysis of gene expression, anti-proliferation from direct cell number counts, and/or subpopulations of drug sensitive or resistant cells.

Those skilled in the art will recognize that mathematical modeling can also be used to consider the entire time course of cell counts in response to multiple concentrations of the one or more anti-cancer drugs or chemotherapeutic agents.

### BRIEF DESCRIPTION OF THE DRAWINGS

FIG. 1 is a diagram of the perfusion bellows pharmacodynamic culture system of the invention. Cells of cancer lines of interest are grown on plastic flakes suspended in a flow-through, bellows-agitated system that allows for homogeneous exposure of cells to drug/drug metabolite solutions and frequent sampling of cells for viability. Direction of arrow indicates the flow of influx/efflux medium.

FIG. 2A is a diagram of a mathematical model that assumes two populations of cells in different states of the cell cycle: cells that are preparing for replication (state 1) and cells that are immediately “pre-replication” (state 2). Cells transition from state 1 to state 2 by a first-order growth rate constant, while replication from state 2 to state 1 is assumed to be fast. FIG. 2B is a diagram of a pharmacodynamic model. This model assumes that the drug(s) act on independent pathways and includes a small antagonistic term. The observed effect of the combination is larger than a model which assumes both drugs acting on the same pathway would predict.

FIG. 3 is a series of graphs showing that tetrac induces anti-proliferation in cancer cells. FIG. 3A shows U87MG cells and FIG. 3B shows MDA-MB cells treated daily with tetrac (10⁻⁹ to 10⁻⁵ M). In FIG. 3C, MDA-MB cells were treated daily with different concentrations of tetrac (10⁻⁸ to 10⁻⁵ M). Cells were harvested at the time points indicated. The total cell numbers taken after each treatment were used as indicators of tetrac-induced anti-proliferation.

FIG. 4 is a series of graphs showing the time course distribution of [¹²⁵I]-tetrac in human glioblastoma U87MG cells. In FIG. 4A, cells were seeded in 100 mm Petri dishes and refed MEM containing 0.25% stripped serum for 2 days. [¹²⁵I]-tetrac (0.227×10⁻⁶M) was added to Petri dishes at time points indicated. The final concentration of [¹²⁵I]-tetrac was 2.27×10⁻¹¹M. Cells were harvested immediately after final tetrac adding. Cytosolic and nuclear proteins were separated as described previously. 10 μl of media, cytosolic and nuclear proteins were prepared for scintillation counting. The results are the average of four experiments. In FIG. 4B, the distribution of [¹²⁵I]-tetrac in cytosolic and nuclear fractions is shown.

FIG. 5 is a series of graphs showing that tetrac and nano-tetrac induce antiproliferation. FIG. 5A shows MDA-MB cells that were treated daily with different concentrations of tetrac and nano-tetrac (2.5×10⁻⁶ and 10⁻⁶ M). FIG. 5B shows MDA-MB cells that were treated daily with different concentrations of tetrac nano (10⁻⁹ to 10⁻⁶ M). Cells were harvested at the time points indicated. Total cell numbers taken after each treatment were used as indicators of tetrac and nano-tetrac-induced anti-proliferation. In FIG. 5C, U87MG cells were treated daily with different concentrations of tetrac or nano-tetrac (10⁻⁹ to 10⁻⁶ M). Cells were harvested at the time points indicated. Total cell numbers taken after each treatment were used as indicators of tetrac or nano-tetrac-induced anti-proliferation.

FIG. 6 is a series of graphs showing the effects of tetrac and nano-tetrac in human adenoid cystic carcinoma (TGS112T) cells. In FIG. 6A, TGS112T cancer cells were treated with 10⁻⁷M T₄ in the presence or absence of 10⁻⁷M tetrac for 24 h. T₄-induced PCNA expression was inhibited by tetrac. In FIG. 6B, TGS112T cancer cells were treated with 10⁻⁷ M T₄ in the presence or absence of 10⁻⁷ M tetrac or nano-tetrac, daily and 1 μCi [³H]-thymidine (final concentration, 13 nM) was added for 24 h. T₄ increased thymidine incorporation, which was inhibited by tetrac and nano-tetrac. Tetrac itself increased thymidine incorporation slightly. In FIG. 6C, TGS112T cells were treated daily with 10⁻⁶ M tetrac or nano-tetrac. Cells were harvested at the time points as indicated. Total cell numbers taken after each treatment were used as an indicator for tetrac- or nano-tetrac-induced anti-proliferation.

FIG. 7 is a series of graphs showing that tetrac induces apoptosis in MDA-MB cells. MDA-MB cells grown in perfusion bellows cell culture were treated with different concentrations of tetrac (10⁻⁷ M to 10⁻⁵ M) daily. Cells were harvested on the day indicated. Two million cells of each sample were prepared for flow cytometry. Flow cytometry was conducted as described in Example 1, infra.

FIG. 8 shows the expression of pro-apoptotic genes by tetrac and nano-tetrac. U87MG human glioblastoma cells (FIG. 8A) and MDA-MB-231 breast cancer cells (FIG. 8B) were treated daily with 10⁻⁶ M of tetrac or nano-tetrac in the bellows perfusion culture system. Cells were harvested after 2 days of treatment and total RNA was extracted. RT-PCR was conducted as described in Example 1, infra. Nano-tetrac significantly stimulated (P<0.02) the expression of pro-apoptotic genes (p53, BAD, PIG3, p21) (see el-Deiry, 1998. Semin Cancer Biol. 8:345-57; Yang et al., 1995. Cell 80:285-91) in U87MG cells, while unmodified tetrac was effective only as an inducer of expression of c-jun. The results in MDA-MB-231 cells were different, in that tetrac enhanced the expression of c-jun, c-fos, and p21 (each, P<0.05 vs. control) to a moderate degree. Nano-tetrac induced expression of BAD (P=0.001), PIG3 (P=0.037), and p21 (P=0.05). Together, results in the figure demonstrate the variable nature of responses to tetrac in the two cell lines and a more consistent response of each cell line to nano-tetrac.

FIG. 9 is a series of graphs showing the comparison of nano-tetrac and tetrac on cell proliferation in non-malignant cells. CV-1 cells (FIG. 9A) and 293 T cells (FIG. 9B) were treated daily with 10⁻⁶ M tetrac or nano-tetrac. Cells were harvested at the time points indicated. Total cell numbers taken after each treatment were used as an indicator for tetrac- or nano-tetrac-induced anti-proliferation.

FIG. 10 is a graph showing the effect of tetrac and cetuximab-induced anti-proliferation. Human breast cancer MDA-MB cells were treated daily with 0.1 μg/ml of cetuximab, tetrac (10⁻⁷ M), or the combination in the bellows perfusion culture system. Cells were harvested at the time points indicated. Cetuximab, tetrac, or combination-induced anti-proliferation was determined by cell number count.

FIG. 11 is a graph showing that the combination of cetuximab and nano-tetrac shows a larger anti-proliferative effect than either drug on its own in colon carcinoma cells.

FIG. 12A is a graph showing the total cell counts different concentrations of cetuximab, nano-tetrac, and the combination of cetuximab and nano-tetrac on K-ras mutant colon carcinoma cells. FIG. 12B is a graph showing the total cell counts for 0.2 μg/mL cetuximab plus nano-tetrac in varying concentrations. FIG. 12C is a graph showing the total cell counts for 1.5 μg/mL cetuximab plus 6 nM nano-tetrac. FIG. 12D is a graph showing the total cell counts for 10 μg/mL cetuximab plus varying concentrations of nano-tetrac.

FIG. 13 is a graph showing the drug effect on the growth rate constant from pharmacodynamic modeling for all treatments.

FIGS. 14A and 14B are graphs showing the effects of tetrac, cetuximab, or the combination at varying concentrations on total cell counts for HCT116 cells.

FIGS. 15A-C are graphs showing the results of flow cytometry experiments for apoptosis.

FIGS. 16A and 16B are a series of graphs showing the results of flow cytometry experiments for non-apoptotic cells.

### DETAILED DESCRIPTION

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural references unless the context clearly dictates otherwise. All patents and publications cited in this specification are incorporated by reference in their entirety.

### DEFINITIONS

For convenience, certain terms used in the specification, examples and claims are collected here. Unless otherwise defined, all technical and scientific terms used herein have the same meaning as commonly understood by one of ordinary skill in the art to which this invention pertains.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent”, which is a biologically, physiologically, or pharmacologically active substance (or substances) that acts locally or systemically in a subject.

As used herein, the terms “angiogenesis agent” or “angiogenic agent” include any compound or substance that promotes or encourages angiogenesis, whether alone or in combination with another substance. Examples include, but are not limited to, T3, T4, T3 or T4-agarose, polymeric analogs of T3, T4, 3,5-dimethyl-4-(4′-hydroy-3′-isopropylbenzyl)-phenoxy acetic acid (GC-1), or DITPA. In contrast, the terms “anti-angiogenesis agent” or “anti-angiogenic agent”, as used herein, refer to any compound or substance that inhibits or discourages angiogenesis, whether alone or in combination with another substance. Examples include, but are not limited to, tetrac, triac, XT 199, and mAb LM609. The structures of representative angiogenic and anti-angiogenic agents are provided herein:

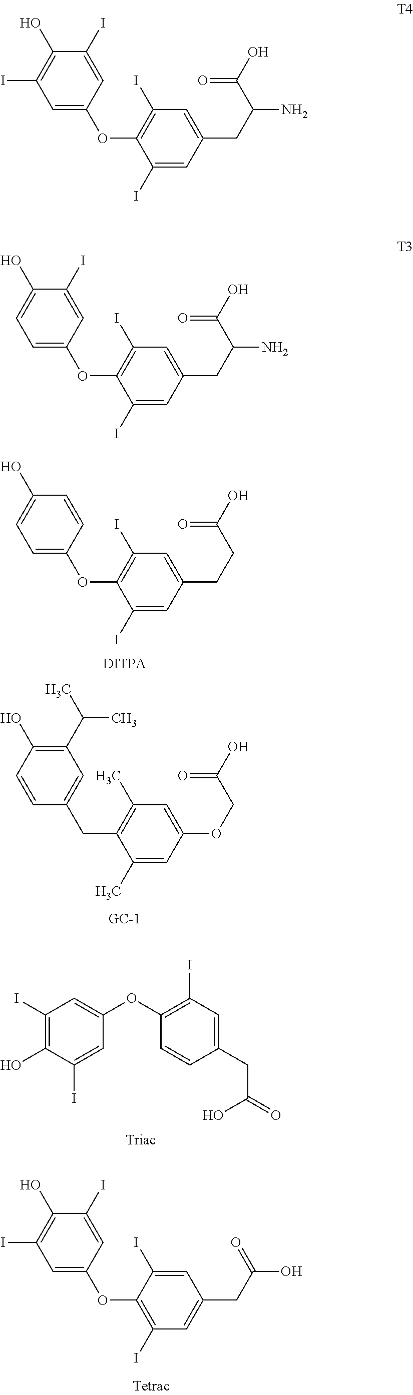 [SMILES: NC(Cc1cc(I)c(Oc2cc(I)c(O)c(I)c2)c(I)c1)C(=O)O ; NC(Cc1cc(I)c(Oc2ccc(O)c(I)c2)c(I)c1)C(=O)O ; O=C(O)CCc1cc(I)c(Oc2ccc(O)cc2)c(I)c1 ; Cc1cc(OCC(=O)O)cc(C)c1Cc1ccc(O)c(C(C)C)c1 ; O=C(O)Cc1ccc(Oc2cc(I)c(O)c(I)c2)c(I)c1 ; O=C(O)Cc1cc(I)c(Oc2cc(I)c(O)c(I)c2)c(I)c1]

A “small molecule” or “small molecule chemical compound” as used herein, is meant to refer to a composition that has a molecular weight of less than 2000 Daltons, preferably less than 1000 Daltons, more preferably less than 750 Daltons. In some embodiments, the small molecule may be less than 500 Daltons. Small molecules are organic or inorganic and are distinguished from polynucleotides, polypeptides, carbohydrates and lipids.

The terms “peptide mimetic”, “mimetic”, or “peptidomimetic” as used herein refer to an agent or a compound that mimics at least one activity of a peptide or compound or a peptide analog in which one or more peptide bonds have been replaced with an alternative type of covalent bond that is not susceptible to cleavage by peptidases.

When referring to a compound, a “form that is naturally occurring” means a compound that is in a form, e.g., a composition, in which it can be found naturally. A compound is “not in a form that is naturally occurring” if, for example, the compound has been purified and separated from at least some of the other molecules that are typically found with the compound in nature. Thus, a “naturally occurring compound” refers to a compound that can be found in nature, i.e., a compound that has not been designed by man. A naturally occurring compound may be harvested from nature or refined from a complex mixture of naturally occurring products or it may be reproduced synthetically.

A “patient,” “individual,” “subject” or “host” refers to either a human or a non-human animal.

The term “modulation” is art-recognized and refers to up regulation (i.e., activation or stimulation), down regulation (i e, inhibition or suppression) of a response, or any combination thereof.

The terms “prophylactic” or “therapeutic” treatment are art-recognized and refer to the administration of one or more drugs or compounds to a host. If administration occurs prior to clinical manifestation of the unwanted condition (e.g., disease or other unwanted state of the host animal), the treatment is prophylactic, i.e., it protects the host against developing the unwanted condition. If administration occurs after the manifestation of the unwanted condition, the treatment is therapeutic, i.e., it is intended to diminish, ameliorate or maintain the existing unwanted condition or other side effects.

The term “mammal” is known in the art and includes humans, primates, bovines, porcines, canines, felines, and rodents (e.g., mice and/or rats).

As used herein, the term “pharmaceutically-acceptable salt” is art-recognized and refers to the relatively non-toxic, inorganic and organic acid addition salts of compounds, including, for example, those contained in the compositions described herein.

The term “pharmaceutically acceptable carrier” is art-recognized and refers to a pharmaceutically-acceptable material, composition or vehicle, such as, for example, a liquid or solid filler, diluent, excipient, solvent or encapsulating material, involved in carrying or transporting any subject composition or component thereof from one organ or portion of the body, to another organ or portion of the body. Each carrier must be “acceptable” in the sense of being compatible with the subject composition and its components and not injurious to the patient. Some non-limiting examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and/or “administered peripherally”, as used herein, are all art-recognized and refer to the administration of a subject composition, therapeutic or other material other than directly into the central nervous system, such that it enters the patient's system and, thus, is subject to metabolism and other like processes.

Likewise, the terms “parenteral administration” and “administered parenterally” are also art-recognized and refer to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and/or intrasternal injection and infusion.

As used herein, “treating” a condition or disease refers to curing as well as ameliorating at least one symptom of the condition or disease.

The term “therapeutic agent” is art-recognized and refers to any chemical moiety that is a biologically, physiologically, or pharmacologically active substance that acts locally or systemically in a subject. This term also refers to any substance intended for use in the diagnosis, cure, mitigation, treatment or prevention of disease or in the enhancement of desirable physical or mental development and/or conditions in an animal or human. For example, the therapeutic agent may be any of the anti-cancer drugs and/or chemotherapeutic agents described herein.

Moreover, the term “therapeutic effect” is art-recognized and refers to a local or systemic effect in animals, particularly mammals, and more particularly humans, caused by a pharmacologically active substance. The phrase “therapeutically-effective amount” means that amount of such a substance that produces some desired local or systemic effect at a reasonable benefit/risk ratio and is applicable to any treatment. The therapeutically effective amount of such substance will vary depending upon the subject and disease or condition being treated, the weight and age of the subject, the severity of the disease or condition, the manner of administration and the like, which can readily be determined by one of ordinary skill in the art. For example, certain compositions described herein may be administered in a sufficient amount to produce a desired effect at a reasonable benefit/risk ratio applicable to such treatment.

The term “synthetic” is art-recognized and refers to production by in vitro chemical or enzymatic synthesis.

Throughout this application, the terms “nanoparticle”, “nanoparticulate” and “nanoparticulate form” are used interchangeably to refer to a modification of any of the active compound(s) of the invention, where the active compound(s) are covalently bound (e.g., by an ester, ether, anhydride, or sulfur linkage) to a polymer wherein the polymer is formulated into a nanoparticle, wherein the active compound is located on the surface of the nanoparticle and wherein the nanoparticle is between 150 and 250 nm in size. For example, conjugation of the anti-angiogenic thyroid hormone analogs via covalent bond to a polymer increases the half life of the compound and/or insures that the compound does not gain access to the interior of the cells (thus, limiting their action to the integrin binding site). The preparation and use of nanoparticulate forms of the anti-angiogenic thyroid hormone analogs are described in the art (see, e.g., WO2008/140507, which is herein incorporated by reference in its entirety). For example, as used herein, the terms “nano-tetrac”, “nano tetrac”, “nanoparticulate tetrac”, “NP-tetrac”, “NP-T”, “tetrac-nano”, “tetrac nano” and the like are used interchangeably to refer to a nanoparticulate form of the thyroid hormone analog tetrac.

### PK/PD Bellows Perfusion System

The use of the in vitro pharmacokinetics/pharmacodynamics bellows perfusion system (“Cancer PK-PD”) described herein will offer data-driven, personalized cancer chemotherapy by a novel in vitro tumor cell assessment system and pharmacokinetic-pharmacodynamic mathematical modeling.

Drusano and co-workers developed an in vitro system for modeling human pharmacokinetics (PK) and pharmacodynamics (PD) for anti-infective agents. (See Drusano, 2004. Nat Rev Microbiol 2:289-300; Bilello et al., 1994. Antimicrob Agents Chemother 38:1386-91). That system employed a semi-permeable hollow fiber by which anti-infective drugs are released in a programmed schedule into medium containing suspended human white blood cells and specific microorganism. The observed killing of microorganisms was mathematically modeled into dose-finding for optimized PD. The Drusano model has been widely applied by the pharmaceutical industry and approved by the FDA for dose-finding in early clinical treatment protocols.

This approach has been optimized for the dosing of new cancer chemotherapeutic agents or of combinations of already-approved cancer treatment drugs against specific cancer cell types, e.g., adenoid carcinoma, colon, head and neck, liver, skin, myeloma, neuroblastoma, ovarian, renal, sarcoma, stomach, thyroid, breast, prostate, lung, pancreas, brain. As described herein, the resulting information can be used to define individual patients' tumor cells' susceptibility to specific drug treatments.

There were several technical problems involved in converting the Drusano hollow fiber system to a system suitable for use in studies of cancer cells. First, cancer cells (except for leukemias) must grow on a fixed surface. Second, cancer cells on such surfaces must optimally be exposed to air and to growth factor-containing medium. Third, the cancer cells must be subject to easy harvesting for counting of cells; assessment of their state of proliferation, e.g., stage of “cell cycle” or entry into the state of programmed cell death (apoptosis); and analysis of the state of expression of specific genes important to cancer cell survival pathways.

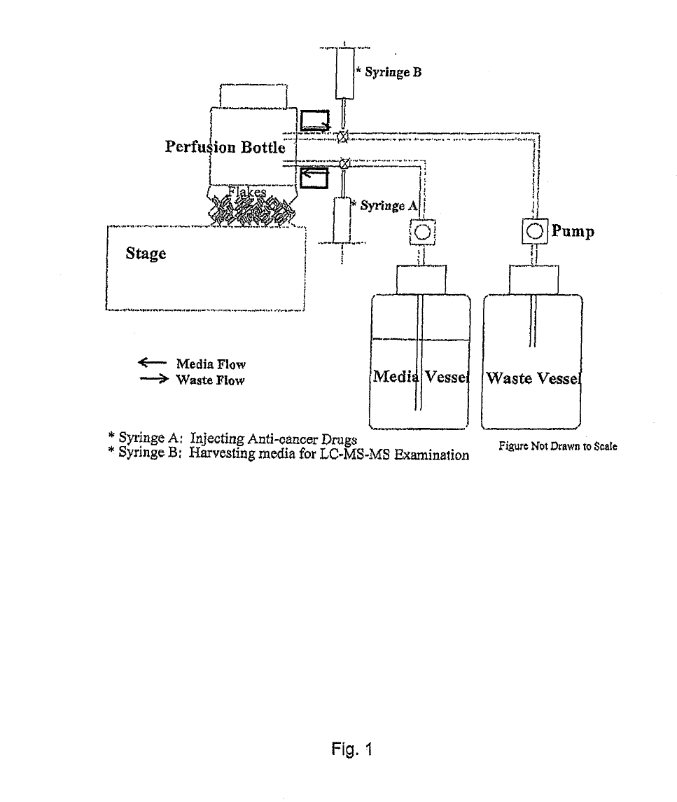

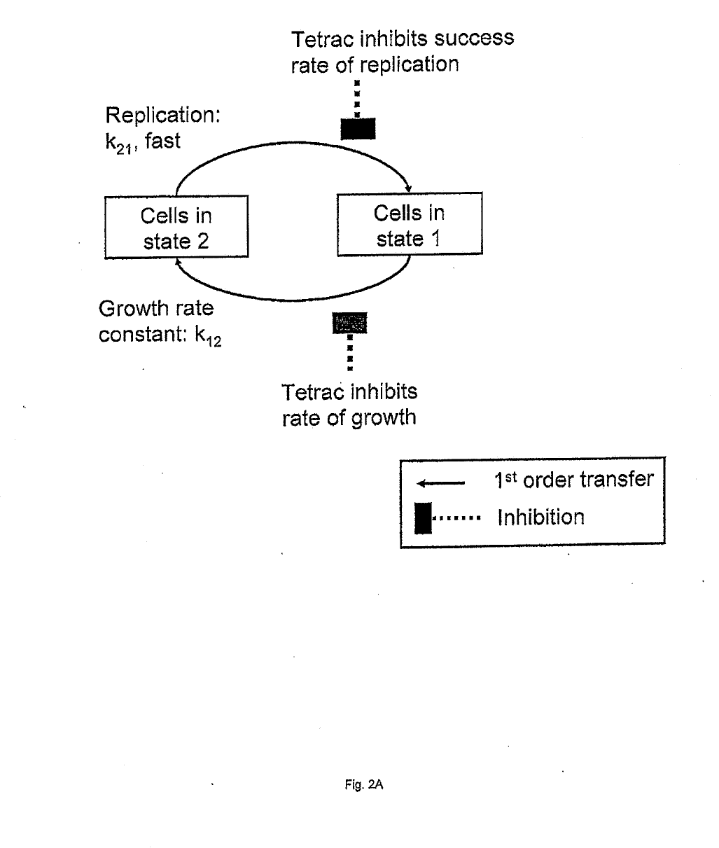

Finding solutions to these technical problems resulted in the development of an in vitro PK/PD system for cancer cells. Specifically, the Drusano hollow fiber paradigm was successfully adapted to cancer cells. (See Lin et al., 2010. J Clin Endocrinol Metab 95(4):1972-80 (Epub Feb. 4, 2010, herein incorporated by reference). Shown in FIG. 1 is a newly developed bellows bottle cell culture system that is a disposable bioreactor capable of high density cell culture for studies of anti-cancer drugs and/or chemotherapeutic agents. This bellows perfusion cell culture system can be used to define in vitro the anti-proliferative pharmacodynamics (PD) and pharmacokinetics (PK) of anti-cancer drugs or chemotherapeutic agents in cancer cells. The system is based on a perfusion (“hollow fiber”) model used to estimate PK and PD of antimicrobial agents (e.g., antibiotics) against epidemiologically important infectious pathogens. The hollow fiber model and perfusion bellows cell culture system allows simulation of concentration-time profiles (PK) expected in humans in an in vitro system and study of the effects over time (PD) of anti-infective and anti-cancer agents in vitro. (See Bilello et al., 1994. Antimicrob Agents Chemother 38:1386-91; Drusano et al., 2002. Antimicrob Agents Chemother 46:464-70; Louie et al., 2009. Antimicrob Agents Chemother 53:3325-30). Such in vitro systems in combination with mathematical modeling can support translation from in vitro to animal models and human clinical trials. The developed pharmacodynamic model describes the full time course of drug effects at various concentrations simultaneously and may be used to predict the effects of other than the studied dosage regimens.

In the Cancer PK/PD bellows perfusion, the standard hollow fiber (dialysis) system has been modified herein by replacing the hollow fiber and chamber paradigm with a flow-through, Bellco perfusion bottle pumping station system (Bellco Biotechnology, Vineland, N.J.). Moreover, in the Cancer PK/PD system described herein, cells of cancer lines of interest are grown on plastic flakes suspended in a flow-through, bellows-agitated system that allows for homogeneous exposure of cells to drug/drug metabolite solutions and frequent sampling of cells for viability.

Each cell culture system has a compressible (bellows) 500 mL bottle that contains cell culture medium and specially-treated polymer flakes to which cells spontaneously attach and proliferate. Through moving bellows and porous membranes, the level of the medium in the bottle changes periodically. Consequently, the cells are alternately submerged in the culture medium and then exposed to 5% CO₂/95% air, which creates a dynamic interface between air and medium on the plated cell surface that maximizes nutrient uptake and oxygen transfer. The system provides a low shear, high aeration and foam-free culture environment for the cancer cells.

Proprietary treatment of the surfaces of the flakes enables seeding and the harvesting of cells and secreted proteins are readily isolated from the perfusate. To establish the cultures, 5×10⁷ cells are seeded in perfusion and non-perfusion bellows bottles and incubated overnight at 37° C. Flakes are then harvested, trypsinized and the cells collected and counted. The number of cells that attach to flakes is 10-15×10⁶ per bottle. For experiments, the perfusion bellows cell culture system is run for 2 days prior to starting experiments. The cell numbers at this point are about 30-50×10⁶ cells/bottle. Cultured cells are then exposed to 1% fetal bovine serum (FBS)-containing medium. Chemotherapeutic agent(s) is/are then added to the medium in the reservoir bottle to achieve the final concentrations reported for each experiment and experiments conducted for 1-21 days. Cells harvested from the polymer flakes may also be subjected to measurements of cell cycling and apoptosis and to analysis of gene expression.

Harvesting as few as ten flakes provides sufficient numbers of cells for analytic purposes, including flow cytometric studies and fluorescence-activated cell sorting (FACS) analysis. Cellular outcomes that are measurable include, but are not limited to, cell cycle arrest, stages of apoptosis, and total cell numbers. The system can also be used to estimate PK and PD of a single new biopharmaceutical cancer chemotherapeutic agent or of a combination of one or more anti-cancer drugs and/or chemotherapeutic agents.

Mathematical modeling is then utilized (see Drusano et al., 2004. Nat Rev Microbiol 2:289-300; Bilello et al., 1994. Antimicrob Agents Chemother 38:1386-91 (incorporated herein by reference)) to increase the amount of information gained from the reported experiments. By considering the entire time course of cell counts in response to multiple concentrations of experimental drug(s) and the control treatment simultaneously, more insight can be gained into the dose-response relationship and the mechanism of action of each drug or drug combination. Also, mechanism-based models such as those described herein are more useful in making predictions, e.g., for other dosage regimens, than empirical growth models are.

In addition, the in vitro Cancer PK-PD system described herein can save animals by decreasing the number of animal studies which need to be conducted, by employing well-defined conditions which allow for investigation of individual factors impacting the PD and permitting the simulation of human pharmacokinetics (PK) based on data from clinical trials.

In the perfusion system cells are exposed alternatively to fresh medium and air. This paradigm optimizes growth conditions for cancer cells by maximizing nutrient uptake and oxygen transfer and supported experiments of up to 3 weeks' duration.

In addition to treating the cells with constant drug concentrations (which mimics in vivo continuous infusion treatment), the in vitro system described herein also allows other dosing regimens to be used. By way of non-limiting example, multiple short-term or intermittent infusions or brief injections can be studied in the bellows perfusion cell culture system by adjusting the flow rate of the medium and the dosing schedule. Likewise, drug concentration/time profiles that are expected or have been obtained in human or animal studies can be simulated and the effects on cancer cells of changing drug concentrations as anticipated in vivo may be observed in the system. Together with mathematical modeling, these in vitro paradigms can support the optimization of design of subsequent animal and human studies, thereby saving time and expense. Moreover, because a wider range of drug concentrations can be studied in vitro than in animal models, selection of appropriate concentrations for in vivo studies may become more efficient.

Those skilled in the art will recognize that cells from relapsed tumors can be subjected to genotyping that has been refined in order to predict aggressiveness and classes of chemotherapeutic agents to which cells may be sensitive. (See Gebauer et al., 2005. Anticancer Res 25:1477-82). Use of this methodology can help to limit the scope of searches in the in vitro Cancer PK-PD System for chemotherapeutic drugs.

### Applications of the Cancer PK-PD System

The continuous cell perfusion model system of the invention provides useful pharmacodynamic information on the application of new drugs or combinations of various agents in vitro to human cancer cell lines. (See Lin et al., PLoS Computational Biology 7(2):e1001073 (2011), which is herein incorporated by reference in its entirety). Additionally, the system can radically change the clinical management approach to individual patients' solid tumors, thereby personalizing therapy and increasing the likelihood of therapeutic success.

The in vitro Cancer PK-PD System individualizes cancer treatment (something that has not been possible previously) and re-structures cancer management. First, the segregation of patients into treatment protocols based broadly on tumor histology and currently available markers is avoided. Second, the likelihood of a response to a particular therapy is importantly increased, which reduces the toxicity and expense of treatments dictated by the protocol, but are unlikely to succeed. Third, protocol changes are defined in behavior and responses to specific chemotherapeutic agents in evolving/relapsing cancers, which thereby enhances likelihood of treatment success.

Those skilled in the art will recognize that exemplary applications of the Cancer PK-PD System of the invention can include, but are not limited to, one or more of the following:

Application to Recurrent Solid Tumors from Individual Patients.

Relapsed cancers are tested in vitro against a panel of single agents, against combinations of agents, and according to various PK models (peak-trough, AUC, etc.) in order to personalize/optimize care of recurrent tumors.

In addition, tumor cells are subjected to RNA microarray before exposures and after exposure in vitro to an optimized regimen to determine which cancer cell survival genes are newly expressed when cells are exposed to specific chemotherapeutic agents or combinations of agents.

Application to Common Primary Tumors that Exhibit Novel Genotypes.

The goal here is to personalize treatment and render maximally effective the treatment choice that is ultimately made.

Application to Rare Solid Tumors for which No Treatment has been Standardized.

The Cancer PK-PD System of the invention can be used to determine an appropriate course of treatment for rare solid tumors.

Application to all Primary Tumors when the System and Insurance Coverages Mature Sufficiently to Permit this Approach.

Those skilled in the art will recognize that the Cancer PK-PD System can also be used to determine an appropriate course of treatment for all primary tumors (not just for recurrent ones).

In Patient-Specific Tumors, Dose-Finding for Existing Effective Cancer Chemotherapeutic Agents with Unfavorable Side Effect Profiles, Including Quality of Life Indicators, when Combined at a Lower Dosage with a Second Agent.

The strategy proposes that dosing that is ordinarily subtherapeutic for an agent with some desirable qualities may be rendered effective in the presence of a second agent. Preliminary studies of this paradigm indicate that two agents in subtherapeutic dosages may achieve certain treatment goals in the model. (See, e.g., Example 2, infra).

Assays of Media of Treated Cells from Patient-Specific Tumors for Release of Vascular Growth Factors.

Many solid tumors release vascular growth factors-vascular endothelial growth factor (VEGF), basic fibroblast growth factor (bFGF), epidermal growth factor (EGF)—whose function is to support an exaggerated blood vessel support system for the tumors. The release of such growth factors may be an indication to combine anti-cancer drug treatment with anti-blood vessel (anti-angiogenic) therapy.

Dose-Finding for Novel Cancer Chemotherapeutic Agents.

The model facilitates the transition in drug development from late preclinical testing into FDA Phase I and early Phase II clinical trials. It may be used to segregate/congregate patients with specific tumor types for clinical trials.

In combination with pharmacodynamic modeling and by including information about the expected pharmacokinetics of a drug, the perfusion bellow cell culture system permits study of the dose-response relationships of anti-neoplastic agents over a very wide concentration range in vitro, and can support translation from in vitro models to animal models and human clinical trials.

Modification of the PK/PD System for Evaluation of Drugs Intended to Treat Leukemias and Lymphomas.

Those skilled in the art will recognize that the system as described here is intended for application to solid tumors. Moreover, technical modifications in the system will be required in order to evaluate cancers that are of hematologic origin, such as leukemias and lymphomas.

Treatment of Cancer Cells with Tetrac or Nano-Tetrac

Integrin αvβ3 has been shown to be expressed on tumor cells, on endothelial and vascular smooth muscle cells, on osteoclasts, and on angiogenically active blood vessel cells. (See Davis et al., 2008. Front Neuroendocrinol 29:211-18; Davis et al., 2009. Am J Physiol Endocrinol Metab 297:E1238-E1246). This restricted expression makes this integrin an attractive target for the development of cancer treatment strategies because nano-tetrac has little effect on proliferation of non-malignant cells. Moreover, the observed additive effects of combinations of nano-tetrac and other chemotherapeutic agents suggests that lower dosages of agents are possible with conjoint therapy.

Tetraiodothyroacetic acid (tetrac) is a deaminated thyroid hormone analogue that binds to the integrin αvβ3 receptor for the hormone. (See Bergh et al., 2005. Endocrinology 146:2864-71; Davis et al., 2006. Cancer Res. 66:7270-75). Tetrac has anti-proliferative activity in cancer cells that are initiated at a cell surface receptor for thyroid hormone on integrin αvβ3. (See Davis et al., 2009. Am J Physiol 297:E1238-E1246; Lin et al., 2009. Am J Physiol 296:C980-C991; Yalcin et al., 2009. Anticancer Res 10:3825-3831; and Glinskii et al., 2009. Cell Cycle 8:3554-3562). Tetrac is pro-apoptotic (see AB Glinskii et al., ibid.). Tetrac is also a polyfunctional anti-angiogenic agent. Tetrac is an antagonist at the receptor, inhibiting binding of agonist L-thyroxine (T₄), and 3,5,3′-triiodo-L-thyronine (T₃) to the integrin of cultured cells (see Berg et al. 2005. Endocrinology 146:2864-71) and blocking nongenomically initiated effects of T₄ and T₃ on signal transduction pathways (see Davis et al., 2006. Cancer Res. 7270-75; Lin et al., 2007. Steroids 72:180-87; and Lin et al., 2008. Carcinogenesis 29:62-69).

Inhibition of the angiogenic action of thyroid hormone by tetrac has been shown in the chick chorioallantoic membrane (CAM) model and in the vessel sprouting model involving human dermal microvascular endothelial cells (HDMEC). Tetrac is effective in the CAM and HDMEC models. This inhibitory action of tetrac is thought to reflect its influence at the RGD recognition site on the integrin, which is relevant to cell surface pro-angiogenic growth factor receptors with which the integrin engages in cross talk and whose activities may be modulated by the integrin.

Tetrac also decreases basic fibroblast growth factor (bFGF) and vascular growth factor (VEGF)-induced angiogenesis in the absence of agonist thyroid hormone analogues (see Davis et al., 2004. Circ Res. 94:1500-1506; and Mousa et al., 2008. Angiogenesis 11:183-90), thereby supporting the hypothesis that there is a cross talk between the integrin and the clustered receptors for VEGF, bFGF, and other pro-angiogenic peptides. Tetrac inhibits thyroid hormone-induced activation of mitogen-activated protein kinase (MAPK, ERK1/2) and cell proliferation in a variety of cancer cell lines (see Davis et al., 2006. Cancer Res. 7270-75; Lin et al., 2007. Steroids 72:180-87; and Lin et al., 2008. Carcinogenesis 29:62-69) and also induces the production of a pro-apoptotic protein, BcL-x short form, in human bronchial adenocarcinoma cells (see Tzirogiannis et al., 2007. Abstract # P1-602 in 89^{th} Annual Meeting, The Endocrine Society), rodent glioma C6 cells (see Lin et al., 2008. Carcinogenesis 29:62-69) and human follicular thyroid cancer cells (see Yalcin et al., 2010. Thyroid 20:281-86). These latter studies suggest that tetrac treatment supports apoptosis in cancer cells, as do RNA microarray observations. (See Glinskii et al., 2009. Cell Cycle 8(21):3544-62; Lin et al., J Clin Endocrinol Metab 95(4):1972-80 (Epub Feb. 4, 2010)).

In addition to the ability of tetrac to block angiogenesis induced by VEGF and bFGF, tetrac has also been shown to enhance the response in vitro to either doxorubicin, etoposide, cisplatin, or trichostatin A of chemotherapy-resistant human tumor cell lines derived from neuroblastoma (SKN-SH/R), osteosarcoma (SaOS2/R), and breast carcinoma (MCF-7/R) cells. (See Rebbaa et al., 2008. Angiogenesis 11:269-76).

Because tetrac is known to have thyromimetic activity within cells (see Lameloise et al., 2001. Eur. J. Endocrinology 144:145-54; Moreno et al., 2008. Thyroid 18:239-253), a plasma membrane-impermeable form of tetrac (i.e., nano-tetrac) has been formulated in which nonreactive nanoparticles are covalently bound to the outer ring hydroxyl group of tetrac (either directly or via a linker). The presence of the nanoparticle does not inhibit the binding of the exposed ligand (tetrac) to the plasma membrane integrin αvβ3. (See Davis et al., Am J Physiol Endocrinol Metab 297:E1238-46 (2009); Glinskii et al., 2009. Cell Cycle 8(21):3544-62).

As described in Example 1, infra, using this system, nano-tetrac was found to inhibit cell proliferation more effectively than tetrac does. Specifically, unmodified tetrac inhibits the proliferation of cancer cells and does so with differing IC50's in different cell lines. When covalently linked to poly (lactic-co-glycolic) acid nanoparticles (PLG), tetrac does not enter the cell, acts exclusively at the cell surface integrin receptor, and suppresses cancer cell proliferation to a greater degree than unmodified tetrac. Moreover, tetrac and nano-tetrac induced apoptosis by suppressing the proliferative activity of thyroid hormone and by differentially affecting expression of anti-apoptotic and pro-apoptotic cells to reduce cancer cell survival.

Likewise, the perfusion bellows system described herein also permitted analysis of treatment of cancer cells with tetrac or nano-tetrac in combination with other chemotherapeutic agents. (See Example 2, infra). For example, both unmodified and nano-tetrac were tested for anti-proliferative efficacy in combination with other anticancer agents such as the commercially-available monoclonal anti-epidermal growth factor receptor (EGFR) antibody Erbitux® (cetuximab). Additive pharmacodynamic results using tetrac in combination with Erbitux® (cetuximab) were observed. Moreover, when tested in an in vitro model, tetrac and nano-tetrac were shown to have favorable pharmacodynamics as anti-cancer agents, whether acting alone or in conjunction with other agents such as cetuximab.

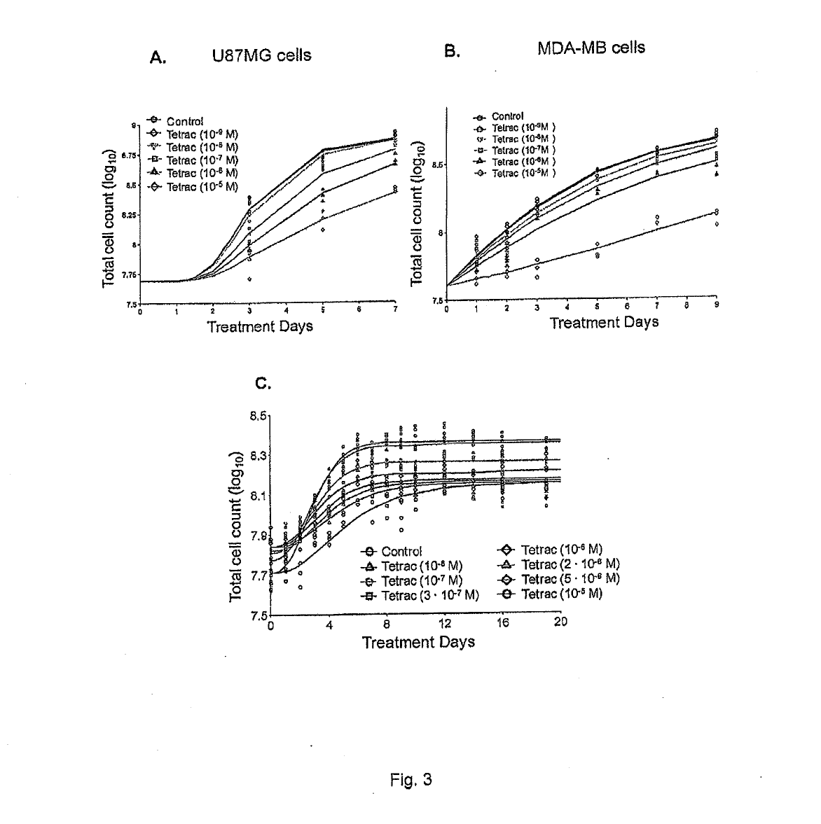

The anti-proliferative effect of tetrac and/or nano-tetrac on cancer cells in the perfusion bellows cell system shown in FIG. 1 was seen as early as 3 days after tetrac or nano-tetrac were added to the system. (See FIG. 3).

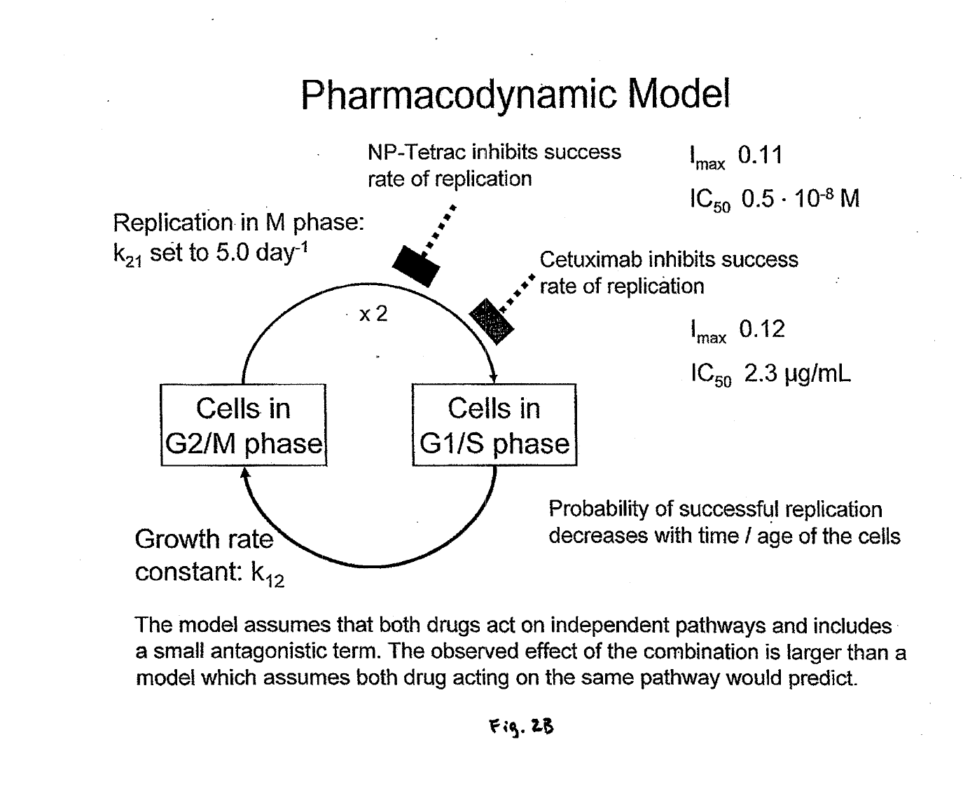

Moreover, in the bellows perfusion cell culture system, the cells are alternately exposed to fresh medium and air. (See FIG. 1). This provides optimized growth conditions for cancer cells by maximizing nutrient uptake and oxygen transfer. Cells can therefore be studied over longer time periods, e.g., up to three weeks as shown in the experiment with tetrac and MDA-MB cells in FIG. 3C and the phase where cell counts have reached a plateau can be observed. By obtaining information about both the slope and the plateau of the cell count with respect to time and by utilizing mathematical modeling (as described in Example 1, infra), two different effects of tetrac on cancer cells can be distinguished. (See FIG. 2).

As described in detail in Example 1, infra, mathematical modeling was utilized to increase the amount of information gained from the reported experiments. By considering the whole time course of cell counts in response to multiple concentrations of tetrac and control treatment simultaneously, more insight can be gained into the dose-response relationship and the mechanism of action of a drug. Also mechanism-based models are more useful in making predictions (e.g., for other dosage regimens) than empirical growth models.

In human breast cancer (MDA-MB) cells two different modeling results are available. For the first model sparse data from two different experiments were pooled (see Table 1, study 1, infra), whereas the second set of parameter estimates is based on a single large experiment with rich sampling (see Table 1, study 2, infra). Therefore the results from study 2 appear more reliable. MDA-MB cells appeared to have similar (or lower) sensitivity to nano-tetrac compared to unmodified tetrac for the effect on growth rate and a higher sensitivity for the effect on success of replication. These mechanism-based models adequately described the cell counts over time and can be used to support the design of future experiments with tetrac and nano-tetrac (or with other anti-cancer cells and/or chemotherapeutic agents).

Inhibition by nano-tetrac of thyroxine-induced MAPK activation and PCNA expression in cultured cells correlates well with the anti-proliferative effects induced by tetrac and nano-tetrac in the perfusion bellows cell culture system. Results of anti-proliferation studies with nano-tetrac analogues in the bellows system are likely to be more predictive of in vivo effects than studies performed in Petri dish cell cultures. The continuous cell perfusion studies provide useful pharmacodynamic information for the application of new drugs to the treatment of a variety of neoplastic diseases.

This, in combination with pharmacodynamic modeling and by including information about the expected pharmacokinetics of a drug, allows the perfusion bellows cell culture system to be used to study the dose-response relationships of anti-neoplastic agents over a very wide concentration range in vitro, and can support translation from in vitro models to animal models and human clinical trials.

The invention will be further illustrated in the following non-limiting examples.

### EXAMPLES

### Example 1

### Pharmacodynamic Modeling of Tetraiodothyroacetic Acid-Induced Anti-Proliferation in Cancer Cells Using a Perfusion Bellows Cell Culture System

### Materials and Methods

Cell Lines.

Human glioblastoma cells (U87MG), human breast cancer MDA-MB-231 cells (MDA-MB), African green monkey kidney epithelial CV-1 cells and human embryonic kidney 293T cells were purchased from ATCC. Human follicular thyroid cancer FTC236 cells were generously provided by Dr. Orlo Clark (University of California at San Francisco-Mt. Zion Medical Center, San Francisco, Calif.). Human adenoid cystic carcinoma cells (TGS112T) were made available from the laboratory of Dr. L. Queimado at the University of Oklahoma. U87MG cells were maintained for study in MEM supplemented with 10% fetal bovine serum (FBS), and MDA-MB, CV-1 and 293T cells were maintained in DMEM that was supplemented with 10% FBS. Follicular thyroid cancer cells were maintained in 50% DMEM/50% Ham's F-12 plus 10 mU/ml of TSH (Sigma). TGS112T cells were maintained in RPMI medium that contained 20 ng/mL epidermal growth factor (EGF) (200 μL of stock at 10 μg/mL), 400 ng/mL hydrocortisone (800 μL of stock at 50 μg/mL) and 5000 ng/mL insulin (50 μL of stock at 10 mg/mL). Cultured cells were maintained in a 5% CO₂/95% air incubator at 37° C.

Pharmacodynamics (PD) of Tetrac.

FIG. 1 shows a newly developed bellows bottle cell culture system that is a disposable bioreactor capable of high density cell culture for studies of anti-cancer drugs. Each cell culture system is a compressible (bellows) 500 mL bottle which contains cell culture medium and specially-treated polymer flakes to which cells spontaneously attach and grow. Through moving bellows and porous membranes, the level of the medium in the bottle changes periodically. Consequently, the cells are alternately submerged in the culture medium and exposed to 5% CO₂/95% air which creates a dynamic interface between air and medium on the cell surface that maximizes nutrient uptake and oxygen transfer. The system provides a low shear, high aeration and foam-free culture environment. Proprietary treatment of the surfaces of the flakes enables seeding and harvesting of cells and secreted proteins are readily isolated from the perfusate.

In a non-perfusion bellows cell culture system that was also used, the medium in each bottle was replaced by fresh medium every 24 h. In the perfusion bellows cell culture system, medium was progressively refreshed over 24 h, i.e., one complete change of medium occurred over 24 h.

In establishing the system, 5×10⁷ cells were seeded in perfusion and non-perfusion bellows bottles and incubated overnight at 37° C. After that, flakes were harvested, trypsinized and cells were collected. Cell numbers were counted. The numbers of cells that were attached to the bellows bottle were 10-15×10⁶ per bottle. For experiments, the perfusion bellows cell culture system was run for 2 d prior to starting experiments. The cell numbers at this point were about 30-50×10⁶ cells/bottle. Cell cultures were then exposed to 1% FBS-containing medium. Tetrac or nano-tetrac was added to the medium in the reservoir bottle to achieve the final concentrations reported for each experiment.

Liquid Chromatography—Tandem Mass Spectrometry (LC/MS/MS)

In LC/MS/MS experiments, medium samples (20 μL) were injected onto an HP 1100 series HPLC system

(Agilent Technologies, Palo Alto, Calif., USA), equipped with a narrow-bore column Zorbax Eclipse XDB-C18 (5 μm, 150×2.1 mm; Agilent). Separation was performed using a mobile phase of 0.1% (v/v) acetic acid (A) and 100% acetonitrile (B), with a linear gradient of 20-60% B over 25 min Flow rate was maintained at 0.2 mL min⁻¹ and elution was monitored by a diode array detector (200-600 nm). The LC effluent was then introduced into a turbo ion-spray source on a Q/STAR-XL quadruple/time-of-flight (TOF) hybrid mass spectrometer (Applied Biosystems, Foster City, Calif., USA). Negative ESI mass spectra were acquired over the range from m/z 100 to 400. The electrospray voltage was set at −4.5 kV and the source temperature was maintained at 475° C. CID spectra were acquired using nitrogen as the collision gas under collision energies of 25-55 V. High purity nitrogen gas (99.995%) was used as the nebulizer, curtain, heater and collision gas source.

Thymidine Incorporation.

TGS112T cells were seeded in 24-well trays and exposed to 10% hormone-depleted FBS-supplemented medium for 2 days, then treated with 0.25% hormone-depleted FBS-supplemented medium prior to starting the experiments. Aliquots of cells were treated with T₄, tetrac or nano-tetrac as indicated, as well as 1 μCi [³H]-thymidine (final concentration, 13 nM) for 24 h. Cells were then washed twice with cold PBS. TCA (5%, 1 mL) was added and the plate was held at 4° C. for 30 min. The precipitate was washed twice with cold ethanol; 2% SDS (1 mL) was added to each well and the TCA-precipitable radioactivity was quantitated in a liquid scintillation counter.

Immunoblotting.

The techniques have been described in a number of publications. (See Davis et al., 2006. Cancer Res. 66:7270-7275; Lin et al., 2007. Steroids. 72:180-187; Lin et al., 2008. Carcinogenesis. 29:62-69; and Davis et al., 2004. Circ Res. 94:1500-1506, each of which is herein incorporated by reference in its entirety). Nucleoproteins were separated on discontinuous SDS-PAGE (9% gels) and the proteins transferred by electroblotting to nitrocellulose membranes (Millipore, Bedford, Mass.). After blocking with 5% milk in Tris-buffered saline containing 0.1% Tween, the membranes were incubated with selected primary antibodies overnight. The secondary antibodies were either goat anti-rabbit IgG (1:1000, Dako, Carpenteria, Calif.) or rabbit anti-mouse IgG (1:1000, Dako), depending upon the origin of the primary antibody Immunoreactive proteins were then detected by chemiluminescence.

RT-PCR.

Total RNA was isolated as described previously. (See Lin et al., 2008. Carcinogenesis. 29:62-69). First strand complementary DNAs were synthesized from 1 μg of total RNA using oligo dT and AMV Reverse Transcriptase (Promega, Madison, Wis.). First-strand cDNA templates were amplified for GAPDH, c-fos, PIG3, c-Jun, and BAD mRNAs by polymerase chain reaction (PCR), using a hot start (Ampliwax, Perkin Elmer, Foster City, Calif.). Primer sequences were GAPDH [5′-AAGAAGATGCGGCTGACTGTCGAGCCACA-3′ (forward) (SEQ ID NO: 1) and 5′-TCTCATGGTTCACACCCATGACGAACATG-3′ (reverse) (SEQ ID NO:2)], c-fos [5′-GAATAAGATGGCTGCAGCCAAATGCCGCAA-3′(forward) (SEQ ID NO:3) and 5′-CAGTCA-GATCAAGGGAAGCACAGACATCT-3′ (reverse) (SEQ ID NO:4)], PIG3 [5′-TGGTCACAG-CTGGCTCCCAGAA-3′(forward) (SEQ ID NO:5) and 5′-CCGTGGAGAAGTGAGGCAGAATTT-3′ (reverse) (SEQ ID NO:6)], c-jun [5′-GGAAACGACCTTCTATGACGATGCCCTCAA-3′ (forward) (SEQ ID NO:7) and 5′-GAACCCCTCCTGCTCATCTGTCACGTTCTT-3′ (reverse) (SEQ ID NO:8)] and BAD [5′-GTT-TGAGCCGAGTGAGCAGG-3′ (forward) (SEQ ID NO:9) and 5′-ATAGCGCTGTGCTGCCCAGA-3′ (reverse) (SEQ ID NO:10)]. The PCR cycle was an initial step of 95° C. for 3 min, followed by 94° C. for 1 min, 55° C. for 1 min, 72° C. for 1 min, then 25 cycles and a final cycle of 72° C. for 8 min PCR products were separated by electrophoresis through 2% agarose gels containing 0.2 μg of ethidium bromide/ml. Gels were visualized under UV light and photographed with Polaroid film (Polaroid Co., Cambridge, Mass.). Photographs were scanned under direct light for quantitation and illustration. Results from PCR products were normalized to the GAPDH signal.

Flow Cytometry Analysis.

Cells were harvested from flakes by trypsinization, washed with PBS and were resuspended in 200 μL PBS (1×10⁵-1×10⁶ cells). To quantify cellular DNA content, cells were permeabilized by fixation with 70% ethanol for 30 min at 4° C. Samples can be stored in 70% ethanol at −20° C. for several weeks prior to propidium iodide (PI) staining and flow cytometric analysis. If cellular DNA quantification was performed on the same day of cell harvest, the cells were washed after permeabilization in PBS and resuspended in 500 μL PBS. Then 2.5 μL RNase (DNase-free) was added to the cell suspension and incubation was carried out at 37° C. for 30 min. The cell suspension was chilled on ice to 4° C. and 50 μL propidium iodide (PI) was added to the cell suspension. Samples were then kept in the dark at room temperature for 30 min, after which they were subjected to flow cytometry. Samples were analyzed on FACSCalibur™ (Becton Dickinson), using CellQuest software to determine DNA content. Fluorescence-activated cell sorting (FACS) analysis was performed using Annexin V-FITC and PI. The relative percentages of cells in G₁, S, or G₂/M phase were calculated from FL-2 histograms using ModFit LT software.

Statistical Methods and Calculations.

Immunoblot and nucleotide densities were measured with a Storm 860 phosphorimager, followed by analysis with ImageQuant software (Molecular Dynamics, Sunnyvale, Calif.). Student's t test, with P<0.05 as the threshold for significance, was used to evaluate the significance of the hormone and inhibitor effects.

Mathematical Modeling.

The time course of cell counts of the various cancer lines treated with different concentrations of tetrac or nano-tetrac was modeled by utilizing a naive pooled approach in NONMEM VI (version 6.2). All time points and treatment arms within each experiment were modeled simultaneously. A mechanism-based model (see Bulitta et al., 2009. Antimicrob. Agents Chemother. 53:46-56) was adapted to describe the proliferation of cancer cells and the inhibition of proliferation by tetrac. This model assumes two populations of cells in different phases of the cell cycle: cells that are preparing for replication (phase 1) and cells that are immediately “pre-replication” (phase 2). Cells transition from phase 1 to phase 2 by a first-order growth rate constant, while replication from phase 2 to phase 1 is assumed to be fast (see FIG. 2).

The number of cells in phase 1 and 2 are described by:

$$\frac{d C 1}{d t} = Rep \cdot InhR \cdot k 21 \cdot C 2 - k 12 \cdot Inhk \cdot C 1$$

$$\frac{d C 2}{d t} = - k 21 \cdot C 2 + k 12 \cdot Inhk \cdot C 1$$

$$Ct = C 1 + C 2$$

where C1 is the number of cells in phase 1, C2 the number of cells in phase 2, k21 the first order rate constant for replication (transition from phase 2 to phase 1), and k12 the first-order growth rate constant for transition from phase 1 to phase 2. The total number of cells Ct is the sum of C1 and C2. Rep is the replication efficiency factor, which is described by:

$$Rep = 2 \cdot ( 1 - \frac{Ct}{Cmax + Ct} )$$

where Cmax is the maximum number of cells. Without tetrac, the replication efficiency factor approaches 2, which reflects a 100% probability of successful replication InhR describes the inhibitory effect of tetrac on the probability of successful replication:

$$InhR = ( 1 - \frac{ImaxR \cdot Tetrac}{IC 50 R + Tetrac} )$$

Where ImaxR is the maximum effect of tetrac on probability of successful replication and IC50R is the tetrac concentration needed to achieve a half-maximal effect.

Inhk describes the inhibitory effect of tetrac on the rate of growth:

$$Inhk = ( 1 - \frac{Imaxk \cdot Tetrac}{IC 50 k + Tetrac} )$$

Where Imaxk is the maximum effect of tetrac on rate of growth and IC50k is the tetrac concentration needed to achieve a half-maximal effect.

Both IC50R and IC50k are measures for the sensitivity of the cancer cells to the effects of tetrac. A low IC50 corresponds to a high sensitivity of the cells to a particular drug effect, and vice versa. While the InhR describes an irreversible removal of cells from the cell cycle, Inhk only slows down the transitioning of cells through the cell cycle. The cells remain in state one for a longer period of time which represents growth and preparation for replication. This is reflected in the decreased slope of the growth curve. (See Lin et al., PLoS Computational Biology 7(2):e1001073 (2011) (incorporated by reference)).

### Results

Tetraiodothyroacetic acid inhibits cancer cell proliferation.

The pharmacodynamics of tetrac as an anti-proliferative agent versus different cancer cells has been studied in the bellows cell culture system shown in FIG. 1.

Human glioblastoma U87MG cells were treated with different concentrations of tetrac (10⁻⁹-10⁻⁵M) for 7 d and tetrac was replenished daily. The turnover rate of tetrac in the culture system was measured. The tetrac concentration detected was 75% of the original concentration after 24 hours of incubation in medium with 10% FBS in the absence of cells at both room temperature and 37° C. There was 12% tetrac decay when tetrac was incubated with cell cultures at 37° C. These results indicate that tetrac is stable in the perfusion bellows cell system.

A model including effects of tetrac on both rate of growth and probability of successful replication (see FIG. 2) and lag-time for growth during the first 2 days adequately described the time course of the cell counts. As shown in FIG. 3A, tetrac caused a concentration-dependent reduction in U87MG cell proliferation. While 10⁻⁹ M tetrac was the least effective, 10⁻⁸ and 10⁻⁷ M tetrac caused more than 15% and 28% decreases in cell counts when compared with the untreated control cells after 7-day treatment. (See FIG. 3A). Higher concentrations of the agent were proportionately more effective. The parameter estimates for IC50k and IC50R (shown in Table 1) suggested that the U87MG cells were more sensitive to the effect on rate of growth than to the effect on success of replication. However the capacity (i.e., the largest possible effect at very high concentrations of tetrac, Imax) was higher for the effect on success of replication (ImaxR>Imaxk).

TABLE 1
Parameter estimates for effects of tetrac on proliferation of cancer cells

| | | Effect on | | Effect on success | |
|---|---|---|---|---|---|
| | | rate of growth | | of replication | |
| | | | IC50k | | IC50R |
| Cell line | Formulation | Imaxk | (μM) | ImaxR | (μM) |
| U87MG | Tetrac | 0.57 | 0.047 | 0.92 | 47.4 |
| MDA-MB 231 | Tetrac (study 1)^{c} | 0.19 | 0.0076 | 0.53^{a} | 4.4 |
| MDA-MB 231 | Tetrac (study 2)^{d} | 0.85 | 5.1 | 0.20 | 0.087 |
| U87MG | nano-Tetrac | 0.34 | 0.0001^{b} | 1^{a} | 0.089 |
| MDA-MB 231 | nano-Tetrac | 1 | 6.3 | 1^{a} | 0.0086 |

^{a}Imax at time = 0, Imax decreases with time (potentially due to functional adaption of the cells or the presence of two or more subpopulations with different sensitivities towards tetrac
^{b}fixed as the lowest concentration studied was 0.001 μM
^{c}sparse sampling, data pooled from two different studies
^{d}rich data from one single study over 19 days

The IC50 estimates for tetrac nanoparticles (nano-tetrac) are hypothetical concentrations assuming all of the tetrac bound on the nanoparticle is available for binding to the integrin receptor.

The anti-proliferative effect of tetrac in human estrogen receptor (ER)-negative breast cancer MDA-MB cells was also studied. Aliquots of cells were treated with several concentrations of tetrac (10⁻⁹-10⁻⁵M) for 9 days and tetrac was replenished daily. Cells were then harvested and counted. The results presented in FIG. 3B reveal a concentration-dependent effect of tetrac on MDA-MB cell proliferation. The highest tetrac concentration (10⁻⁵M) induced a large anti-proliferative effect whereas at lower concentrations tetrac did not show anti-proliferative effects during the earlier days of treatment. (See FIG. 3B). The results shown in FIG. 3B are pooled data from two experiments (one with sampling on days 1 and 2, and one with sampling on days 3, 5, 7, and 9) in MDA-MB cells with the same tetrac concentrations investigated. Modeling suggested a higher sensitivity (lower IC50) for the effect on growth rate and a larger capacity (larger Imax) of the effect on replication. The model assumed a decrease in ImaxR over time, which could be due to functional adaptation of the cells or the presence of two or more subpopulations with different sensitivities towards tetrac.

In another study, MDA-MB cells were treated with 7 different concentrations of tetrac ranging from 10⁻⁸ to 10⁻⁵ M or with control medium for 19 days and total cell counts were determined every one or two days. (See FIG. 3C). A model including effects on both rate of growth and success of replication (see FIG. 2) and a lag-time of growth adequately described the data. The parameter estimates suggest a higher sensitivity (lower IC50) for the effect on probability of successful replication and a larger capacity (larger Imax) of the effect on rate of growth.

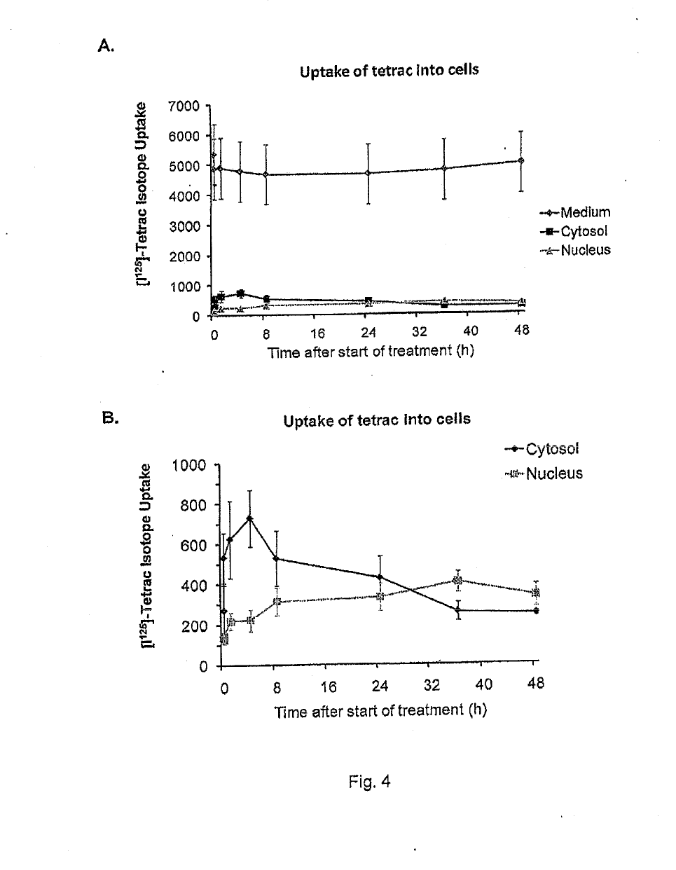

Although tetrac does have a growth-suppressive effect late in the treatment period, it may also have a proliferative effect on cancer cells. This presumably reflects access of the agent to the cell interior where it is a thyroid hormone agonist (thyromimetic), rather than an inhibitor (see Lameloise et al., 2001. Eur J Endocrinology 144:145-154), as it is exclusively at the cell surface receptor. In order to examine whether tetrac enters cells, human glioblastoma U87MG cells were incubated over different time periods with [¹²⁵I]-labeled tetrac. Cells were harvested and medium, plasma membranes, cytosols and nuclear extracts were prepared for scintillation counting. [¹²⁵I]-Labeled tetrac reached the maximal concentration in the cytosolic fraction after 4 hours of incubation and increased in nuclear fractions after 36 hours of incubation. (See FIG. 4). Thus, tetrac enters relatively quickly into cytosol and more slowly into nuclei.

Nano-Tetrac Shows a Consistent Anti-Proliferative Effect in Cancer Cells.

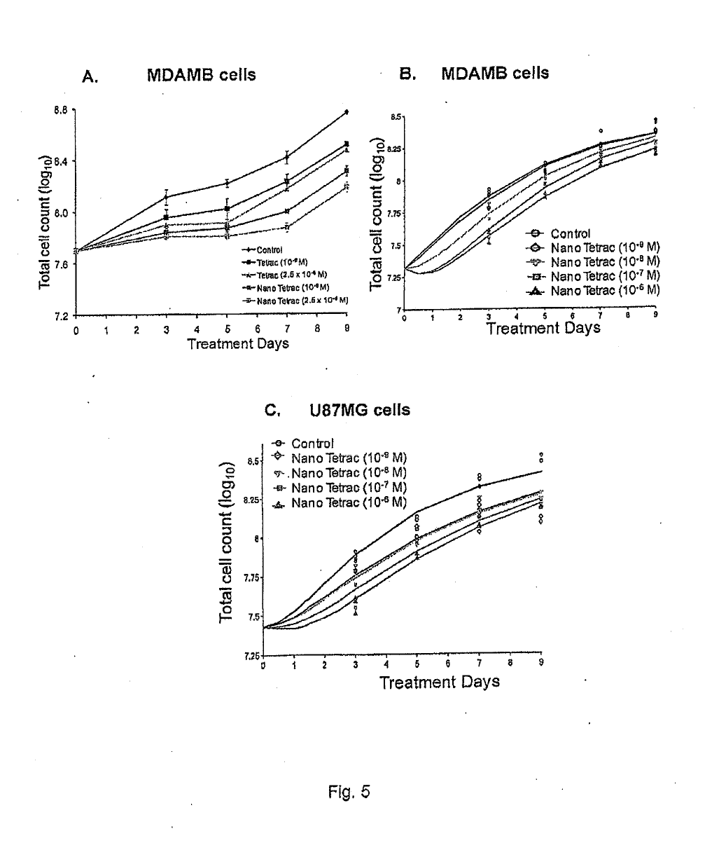

In order to prevent uptake of tetrac by cancer cells, the hormone analogue was reformulated as a poly(lactide-co-glycolide) nanoparticle. The anti-proliferative effect of the resulting tetrac nanoparticles was then studied in MDA-MB cells. Cells were treated with constant concentrations of 10⁻⁶ and 2.5×10⁻⁶ M tetrac or nano-tetrac for 9 d. Results indicate that the anti-proliferative effect of nano-tetrac in MDA-MB cells is more prominent than that of unmodified tetrac. (See FIG. 5A). There was a 100-fold concentration difference in anti-proliferation efficacy between tetrac and nano-tetrac. In another study MDA-MB cells were treated with 4 different concentrations of nano-tetrac (10⁻⁹ to 10⁻⁵ M) for 9 days. (See FIG. 5B). Based on mathematical modeling, the sensitivity of the MDA-MB cells for the nano-tetrac effect on probability of successful replication was considerably higher than that for the effect on rate of growth, while the capacity was similar for both effects (see Table 1, supra).

Studies of nano-tetrac-induced anti-proliferation were also conducted in U87MG cells and results indicate that the anti-proliferative effect of nano-tetrac was concentration-dependent. Concentrations of the nanoparticulate as low as 10⁻⁹M reduced cell number by 36% (control vs. 10⁻⁹ M nano-tetrac=1.641×10⁸ vs. 2.264×10⁸) after 7 days of treatment. (See FIG. 5C). Modeling suggested a higher sensitivity (lower IC50) for the effect on replication and a higher capacity for the effect on rate of growth (see, Table 1, supra). Both IC50k and IC50R were lower for nano-tetrac than for unmodified tetrac in U87MG cells indicating a higher sensitivity to nano-tetrac (see, Table 1, supra). For both MDA-MB cells and U87MG cells, the model assumes a decrease in ImaxR over time and a lag-time of growth during the first two days.

Tetrac and Nano-Tetrac Inhibit Thyroid Hormone-Induced Proliferation of Human Adenoid Cystic Carcinoma Cells.

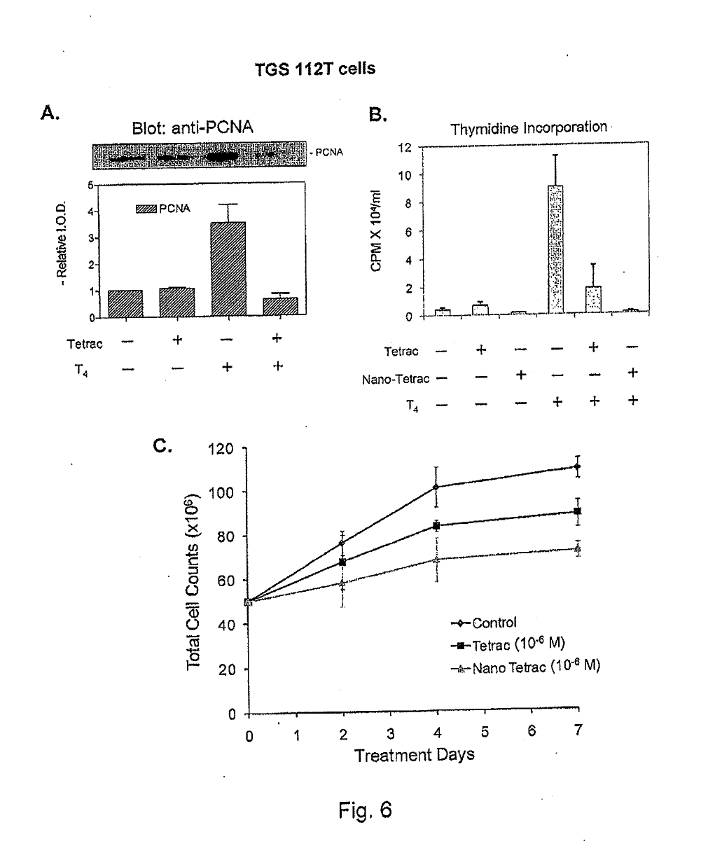

Human adenoid cystic carcinoma TGS112T cells were cultured in medium that contained 0.25% hormone-stripped serum for 2 days and then treated with 10⁻⁷ M tetrac or nano-tetrac for 30 minutes prior institution of 10⁻⁷ M T₄ treatment for 24 hours. Nuclear proteins were separated by SDS-PAGE followed by western blot analysis with proliferating-cell nuclear antigen (PCNA) antibody. Thyroid hormone-induced PCNA accumulation was inhibited by tetrac. (See FIG. 6A). Proliferation of TGS112T cells was stimulated by 10⁻⁷ M T₄, while tetrac at the same concentration had little effect alone, but blocked the T₄ effect. (See FIG. 6A) Inhibition of thyroid hormone-induced proliferation was confirmed by inhibition by tetrac and nano-tetrac of thyroid hormone-induced accumulation and [³H]-thymidine incorporation. (See FIG. 6B). In studies of thymidine incorporation in the same cell line, nano-tetrac alone reduced baseline thymidine incorporation, and reduced T₄-induced thymidine incorporation by more than 14-fold that of tetrac (70-fold reduction vs. 5-fold reduction). (See FIG. 6B). On the other hand, although tetrac inhibited thyroxine-induced thymidine incorporation, unmodified tetrac, when added alone stimulated thymidine incorporation (see FIG. 6B) to a limited degree in this relatively brief study. The effect of nano-tetrac and tetrac on cell proliferation was examined by counting TGS112T cells which were treated daily with 10⁻⁶ M tetrac or 10⁻⁶ M nano-tetrac. The decrease in cell count in the nano-tetrac-treated cells (1.69×10⁸ cells) after 7 days was 2-fold more than that of the tetrac-treated culture (3.65×10⁸ cells), compared to the untreated control cultures. (See FIG. 6C).

Role of Apoptosis in the Tetrac Effect on Cancer Cells.

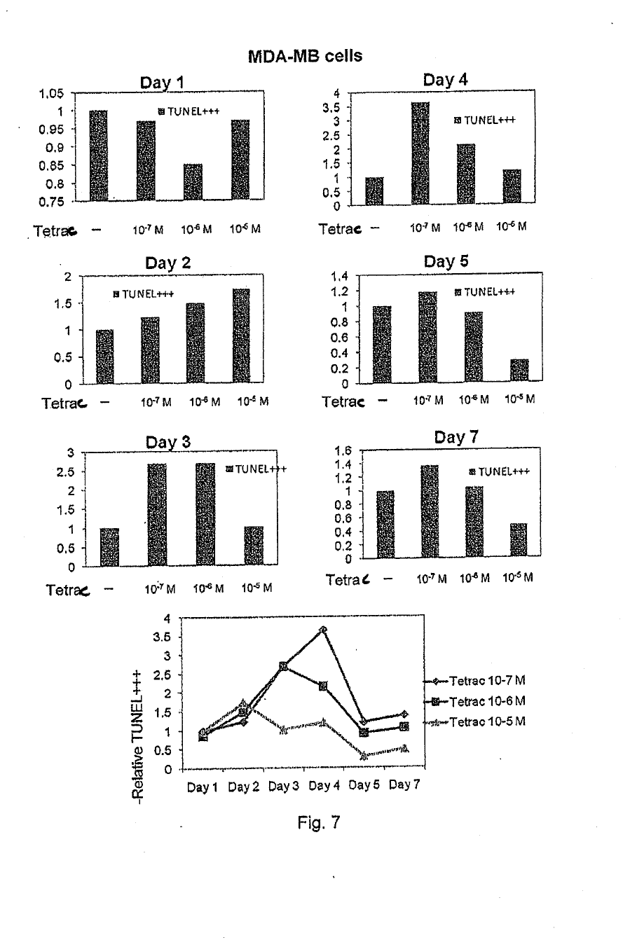

Cells were harvested for FACS analysis 1-5 d after treatment with 10⁻⁷ to 10⁻⁵ M tetrac. There was a 10-fold increase of apoptotic cells with 10⁻⁵ M tetrac treatment as compared to untreated control cells at 1 d and 10-7 M and 10-6 M tetrac also induced apoptosis. (See FIG. 7). By day 2, tetrac, 10⁻⁷M and 10⁻⁶ M, induced apoptosis by 2.5-fold as compared to untreated control. Similar results were obtained after 4 days of treatment of tetrac. That is, cells treated with 10⁻⁷ M tetrac showed highest proportion of apoptotic cells. (See FIG. 7). These results raise the possibility that there are two dose-dependent types of tetrac-induced apoptosis: one is induced transiently by 10⁻⁵ M tetrac and the other is induced by 10⁻⁶ and 10⁻⁷ M tetrac.

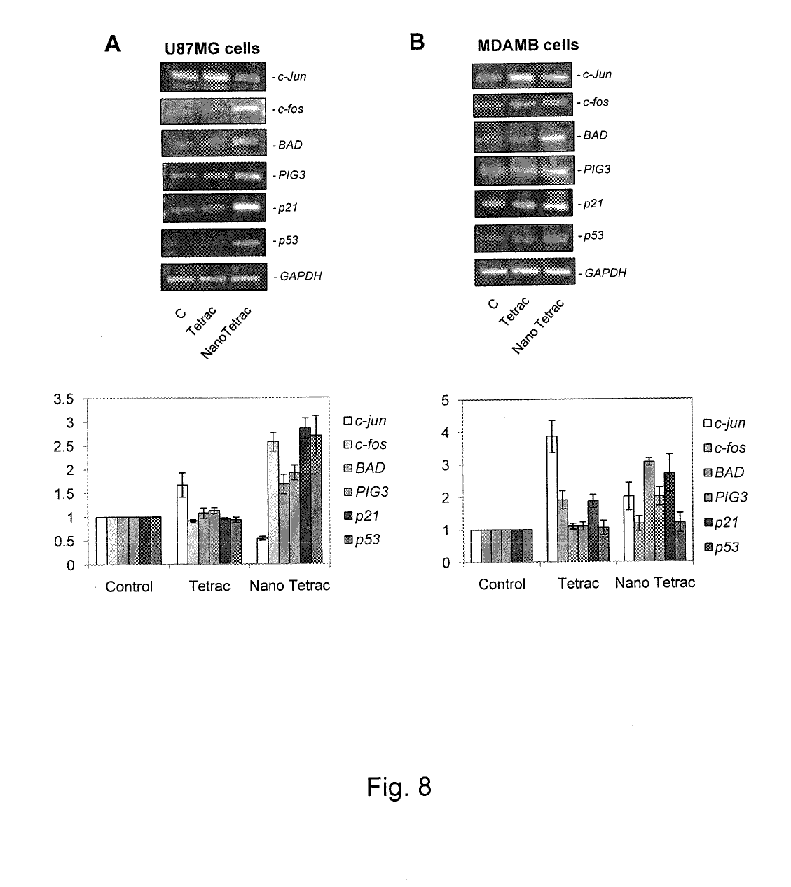

The pro-apoptotic gene expression in tetrac- and nano-tetrac-treated MDA-MB cells and U87MG cells was also examined. RNA was extracted from cells harvested from the perfusion bellows cell culture system at the end of treatment. Primers for PIG3, c-jun and c-fos were constructed for RT-PCR studies. Treatment of cells for 1 day with tetrac (10⁻⁶ M) increased expression of c-fos and c-jun. (See FIG. 8). Nano-tetrac increased expression of PIG3, c-fos and c-jun. (See FIG. 8).

Effects of Tetrac and Nano-Tetrac on Non-Malignant Cells.

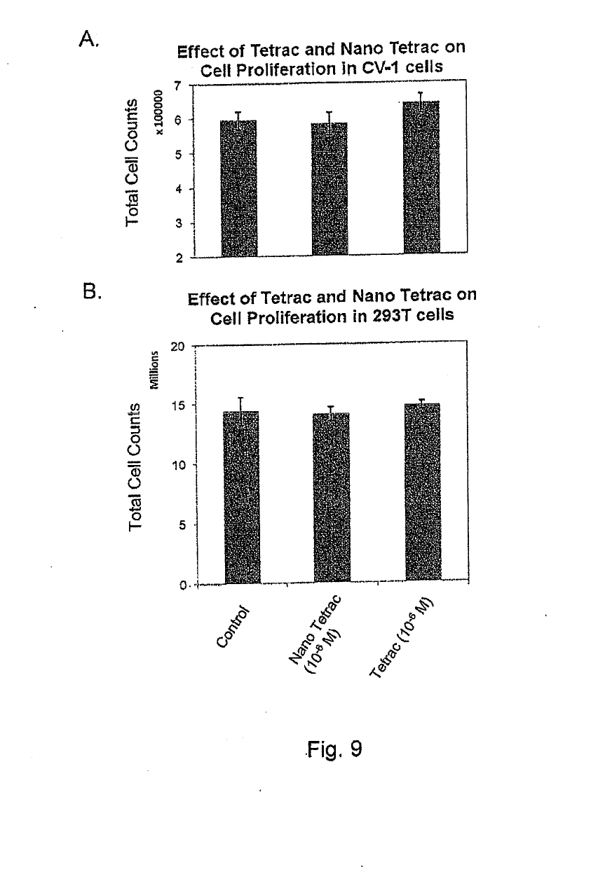

In order to confirm that tetrac and nano-tetrac only suppress cell proliferation in cancer cells, tetrac and nano-tetrac were also examined for potential anti-proliferative effects on immortalized non-malignant cells, using monkey kidney epithelial CV-1 cells and human embryonic kidney 293T cells. The cells were treated daily with either 10⁻⁶M tetrac or 10⁻⁶M nano-tetrac for 7 days, then were harvested, counted and examined microscopically. There was no significant change in either cell numbers (see FIG. 9) or in morphology comparing the untreated control cells and those treated with either tetrac or nano-tetrac. These results suggest that nano-tetrac and tetrac only affect malignant cell proliferation and not that of non-neoplastic cells.

Tetrac Potentiates Cetuximab-Induced Apoptosis in Human Breast Cancer Cells.

The EGFR antibody, cetuximab, has been used in clinical trials either alone or combination with other anti-cancer drug. Cetuximab inhibits binding of the endogenous ligand for the receptor (EGF) and decreases cell motility, invasiveness and metastasis and also promotes apoptosis. (See Kalofonos et al., 2006. Curr. Top. Med. Chem. 6:1687-1705 (incorporated herein by reference)). Combining cetuximab with various chemotherapeutic agents has revealed additive or potentiated growth inhibition in various cancer cell lines, such as those from colon, head and neck, breast, kidney and bladder. (See Balin-Gauthier et al., 2006. Cancer Chemother. Pharmacol. 57:709-718; Martens et al., 2008. Clin. Cancer Res. 14:5447-5458). Approved for clinical use against head-and-neck and colorectal cancers, cetuximab has recently been shown to be effective against invasive glioblastoma xenografts in the mouse. (See Prichard et al., 2007. Laryngoscope. 117:674-679).

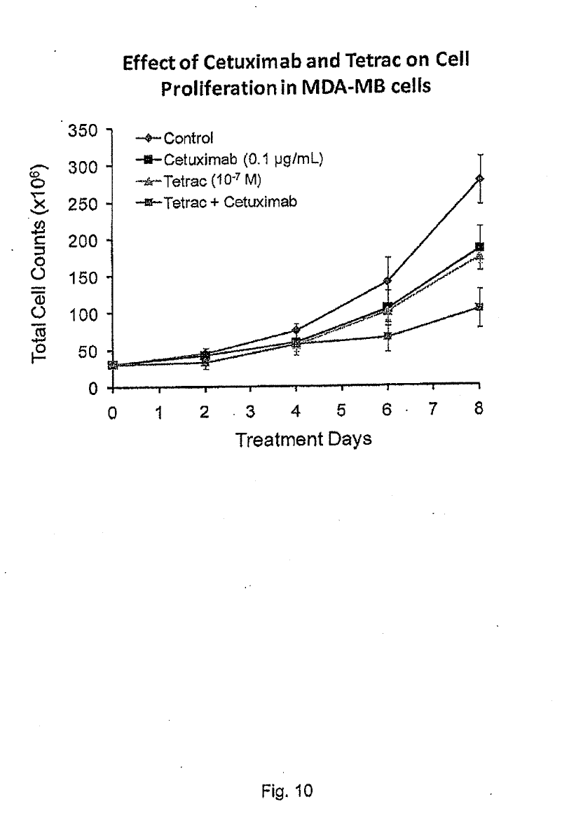

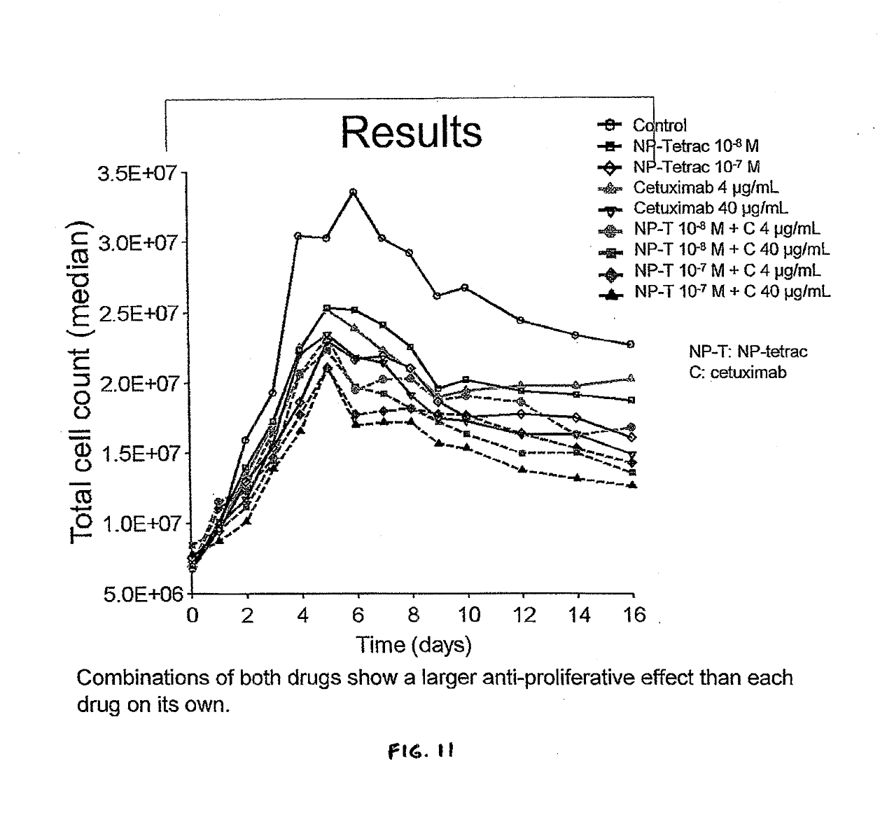

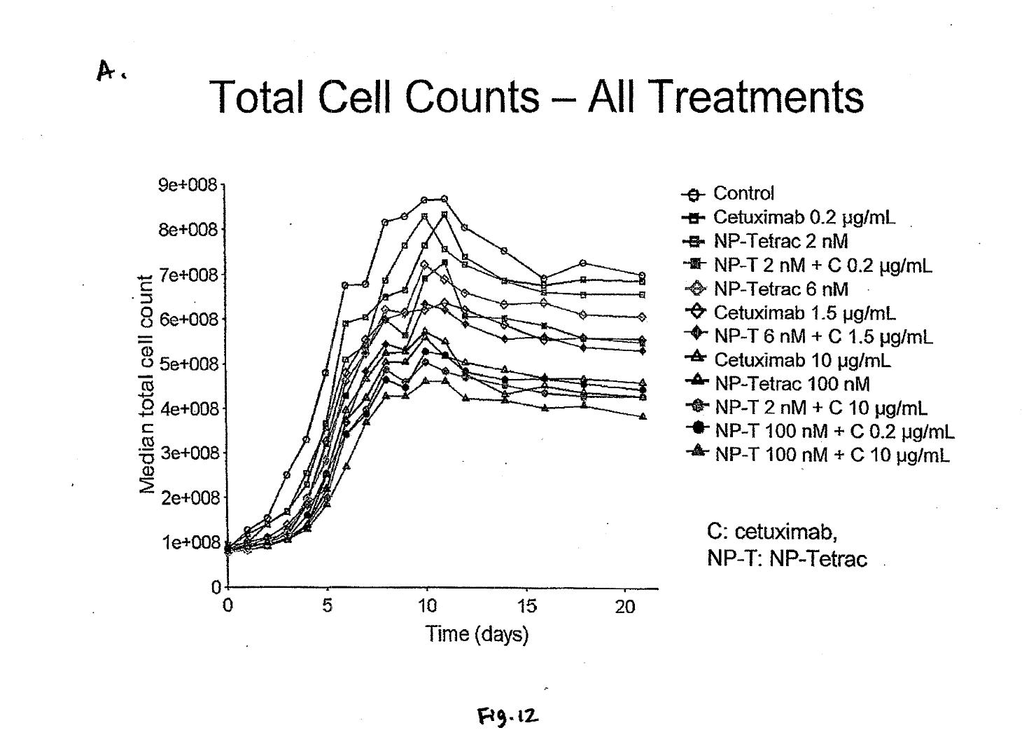

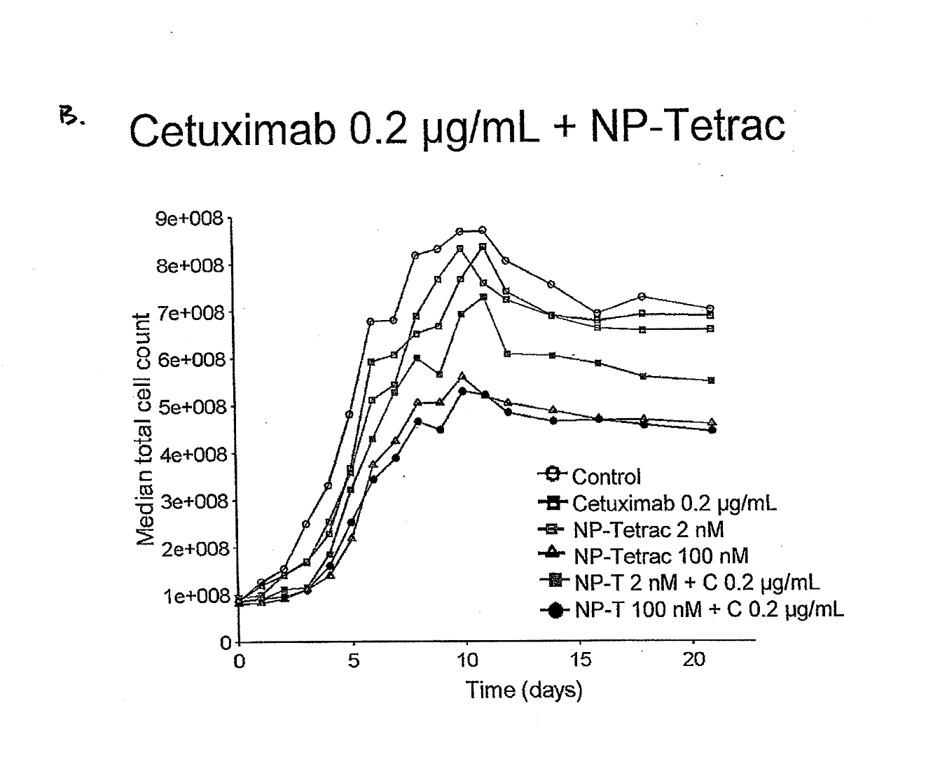

In order to examine whether tetrac or nano-tetrac potentiate cetuximab-induced anti-proliferation, MDA-MB cells were treated with cetuximab (0.1 μg/ml) in the presence or absence of 10⁻⁷ M tetrac. Both agents suppressed cell proliferation in MDA-MB cells. (See FIG. 10). The inhibitory effects on cell growth of cetuximab and tetrac after 8 days treatment were 34% and 38%, respectively. The combination of tetrac and cetuximab reduced total cell number by up to 63%. These results suggest that the combination of tetrac and cetuximab may increase efficacy of cancer chemotherapy when compared to effects of either drug alone.

### Example 2

### Anti-Proliferative Effects of Erbitux® (Cetuximab) and Tetrac Nanoparticles on Colon Cancer Cells

Using the bellows perfusion cell culture system shown in FIG. 1, the effects of tetrac nanoparticles (NP-Tetrac) plus cetuximab on proliferation of colon cancer cells in the flasks were examined.

Effects of NP-Tetrac Plus Cetuximab on Proliferation of Colon Cancer Cells.

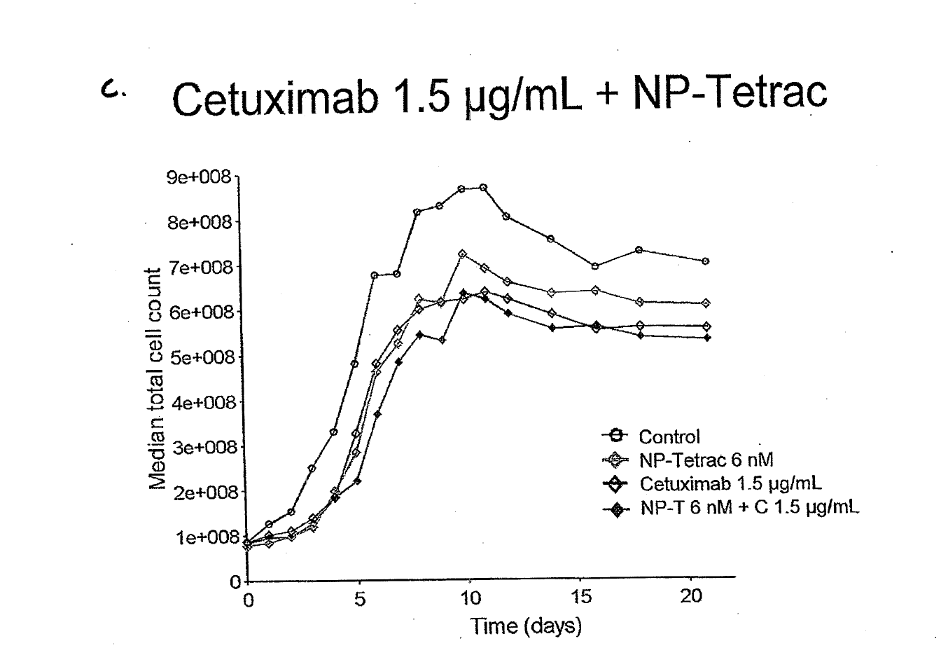

Cells were grown on specially treated flakes in cell culture flasks. The cell culture medium contained 10% fetal bovine serum and various concentrations of cetuximab and NP-Tetrac. The medium was refreshed every 24 hours. The results of these studies are shown in FIG. 11, which demonstrates that the combinations of both drugs showed a larger anti-proliferative effect than each drug on its own.

Effects of NP-Tetrac Plus Cetuximab on Proliferation of Colon Cancer Cells in the Bellows Perfusion System.

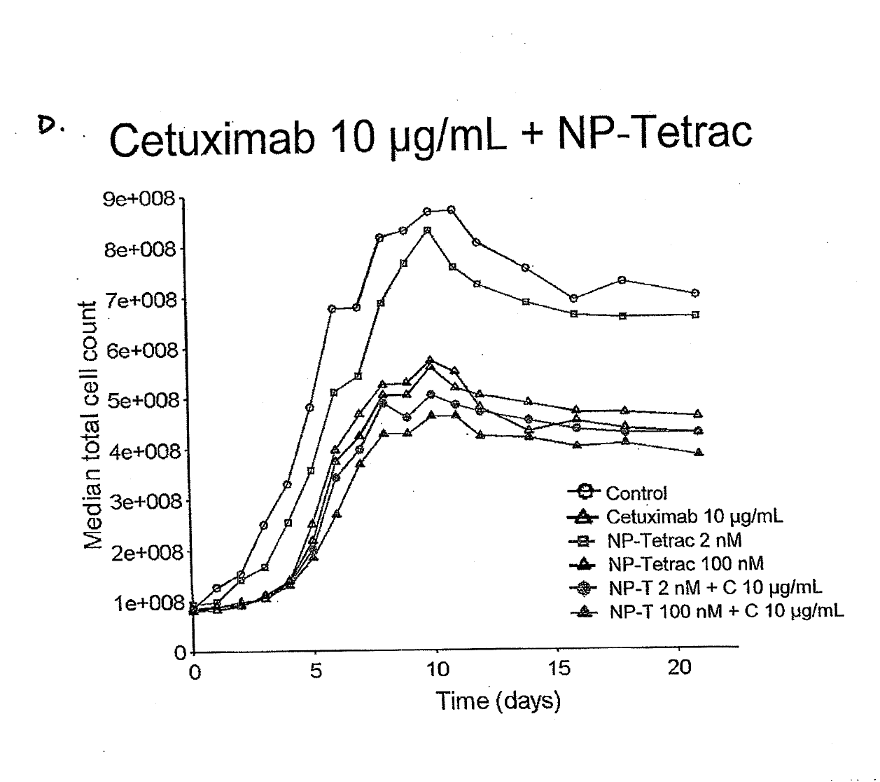

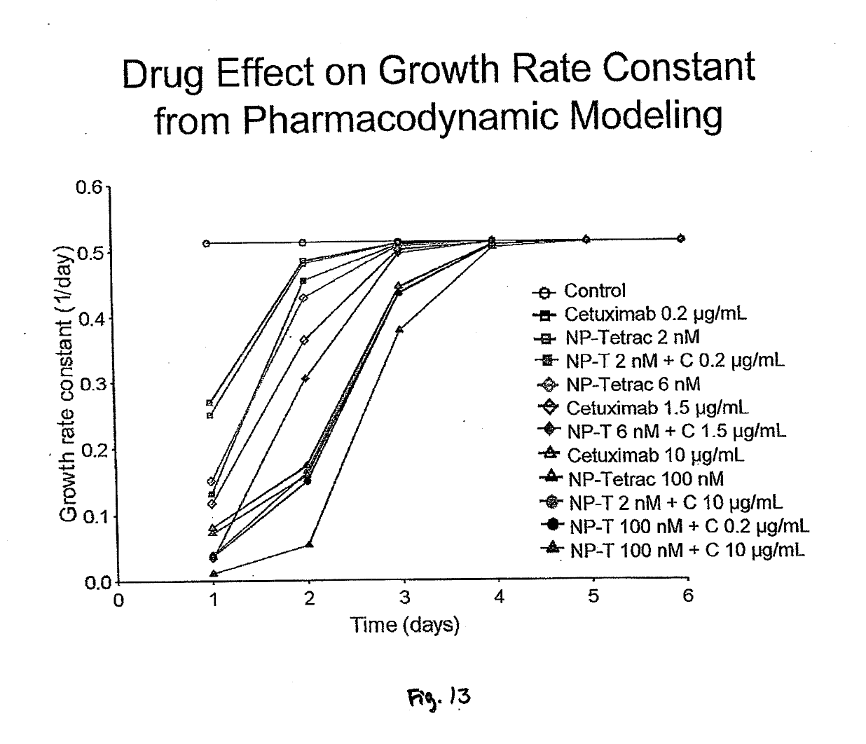

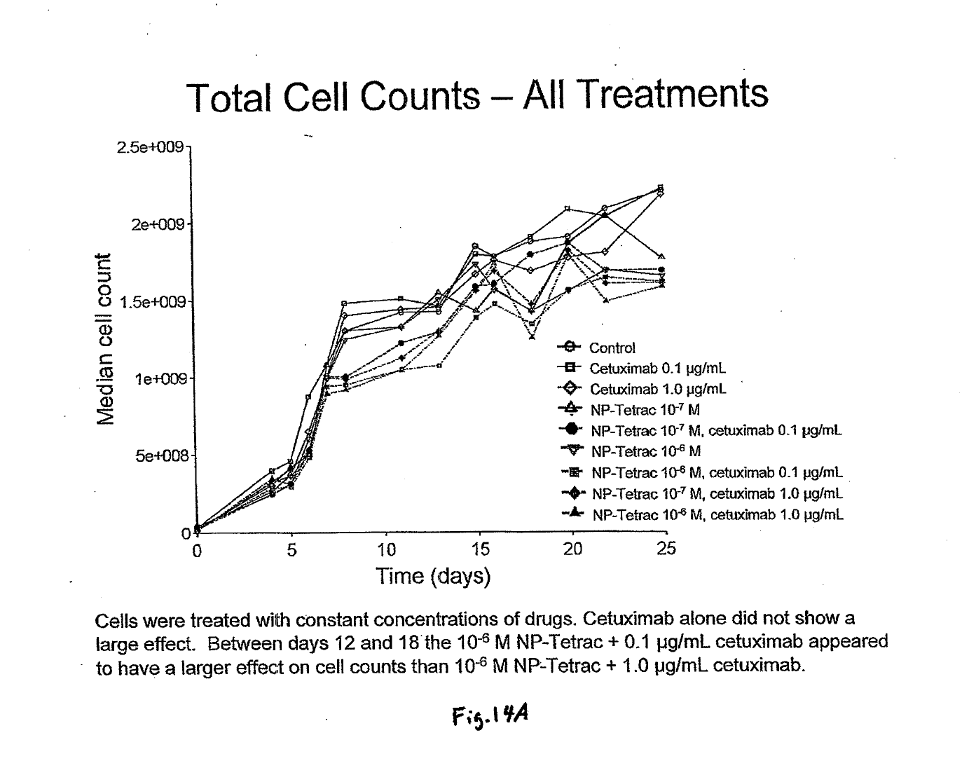

Cells were grown on specially treated flakes in the bellows perfusion cell culture system. The cell culture medium contained 10% fetal bovine serum and various constant concentrations of cetuximab and NP-Tetrac. The system was constantly perfused by fresh medium. The results are shown in FIGS. 12-13.

### Results

Mathematical modeling (see FIG. 2B) suggests that the growth rate constant is decreased in a concentration-dependent manner during the first five days of treatment. Additional effects might be present but likely were not large enough to be identified in this model. The model shown in FIG. 2B, which assumes independent pathways of action for the two drugs (NP-Tetrac and cetuximab) provided adequate fits to the data.

The parameter estimates used herein are as follows:
Cetuximab: Iₘₐₓ 0.86, IC₅₀ 0.01 μg/mL
NP-Tetrac: Iₘₐₓ 0.87, IC₅₀ 0.08 nM

Effects of NP-Tetrac Plus Cetuximab on Proliferation and Viability of K-Ras Mutant (HCT116) Cells.

Approximately 50% of colon cancer patients in a clinical trial showed resistance to cetuximab. (See Jonker et al., 2007. N Engl J Med 357:2040-48). HCT116 cells, a fast growing a malignant cancer cell, have a mutation in the K-ras protooncogene and are resistant to cetuximab.

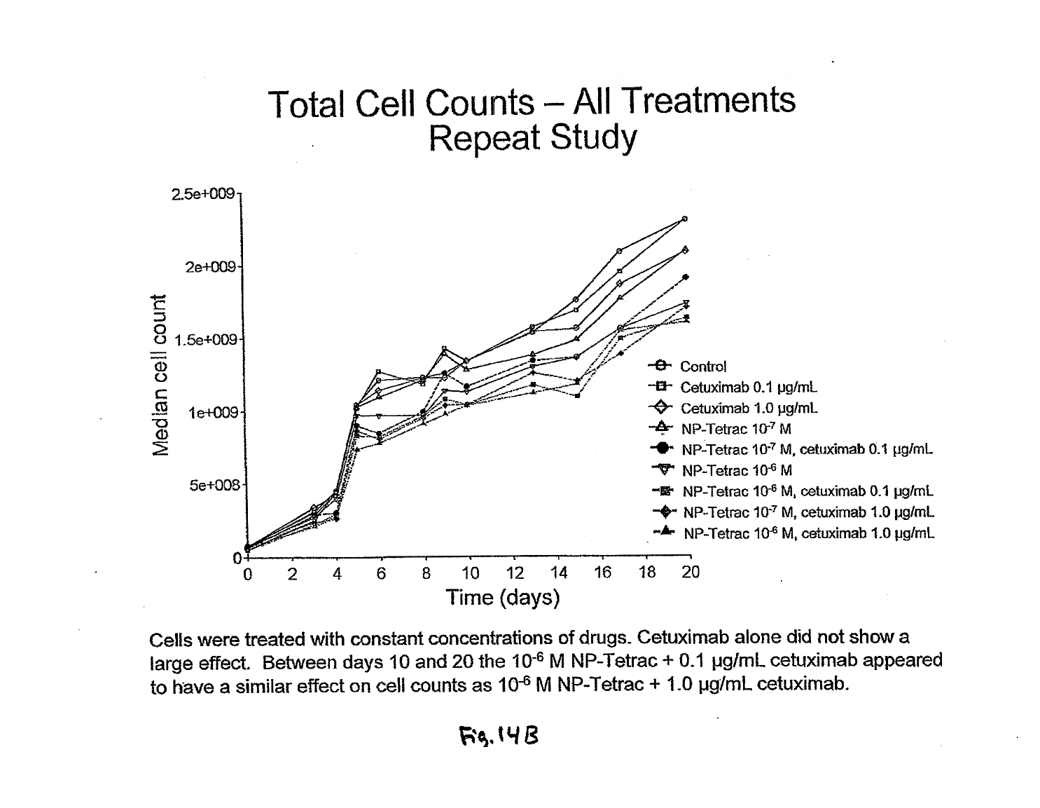

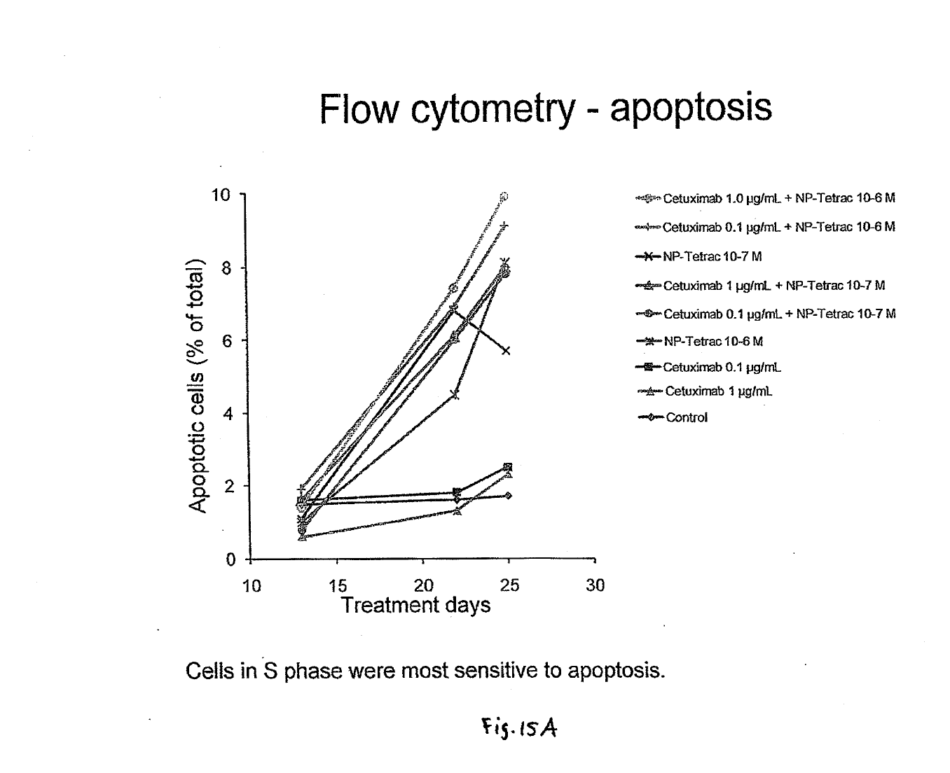

In the experiments described herein, cells were treated with a constant concentration of drugs. Cetuximab alone did not show a large effect. In one experiment, between days 12 and 18, the 10⁻⁶M NP-Tetrac+0.1 μg/mL cetuximab appeared to have a larger effect on cell counts than 10⁻⁶M NP-Tetrac+1.0 μg/mL cetuximab. In another experiment, between days 10 and 20, 10⁻⁶M NP-Tetrac+0.1 μg/mL cetuximab appeared to have a similar effect on cell counts as 10⁻⁶M NP-Tetrac+1.0 μg/mL cetuximab. (See FIGS. 14A and 14B).

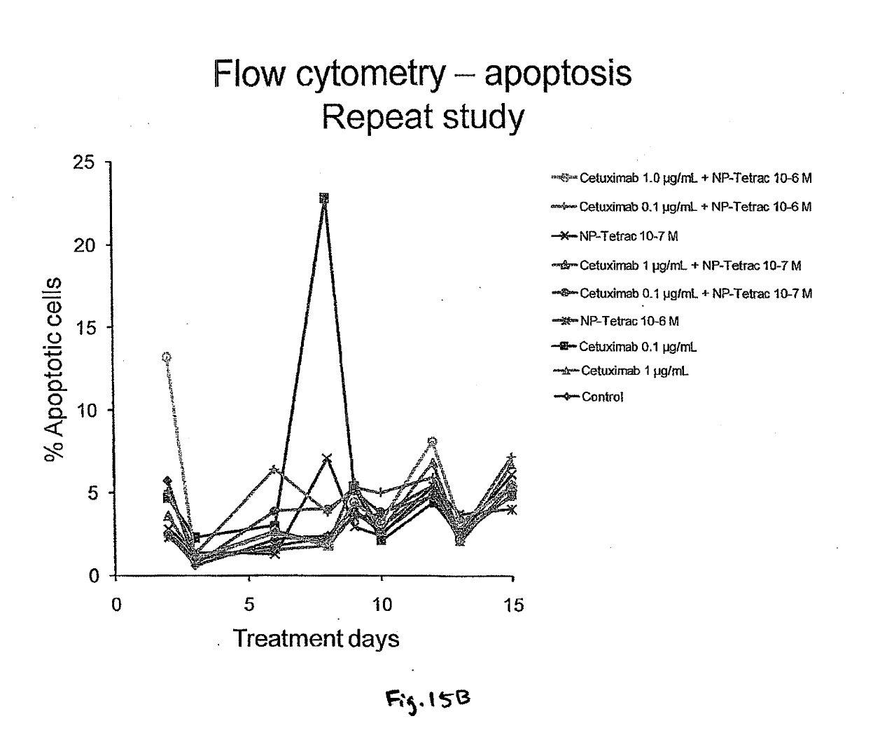

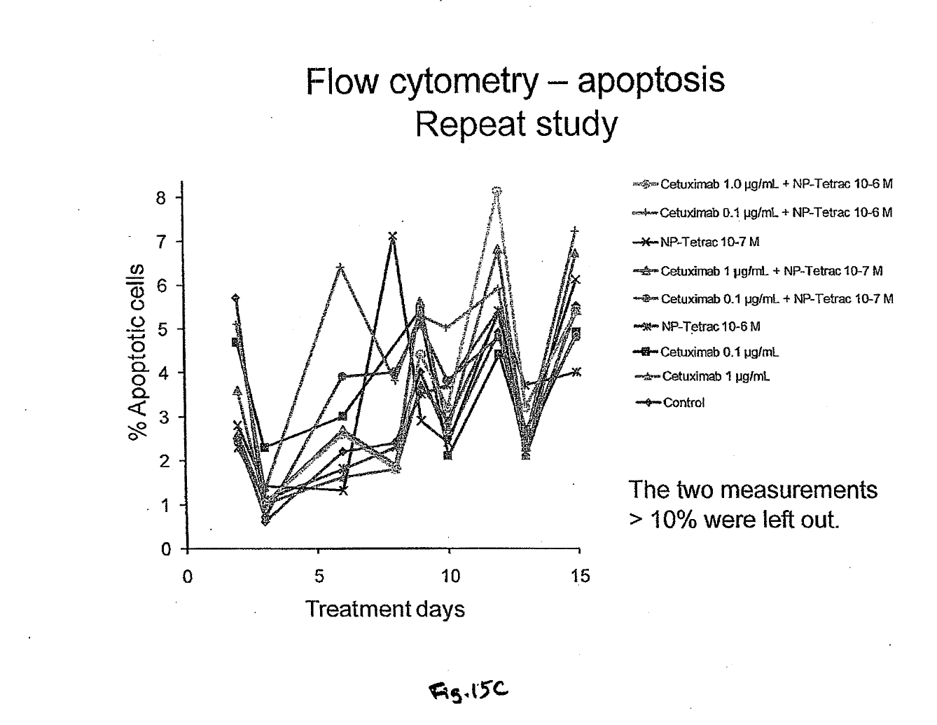

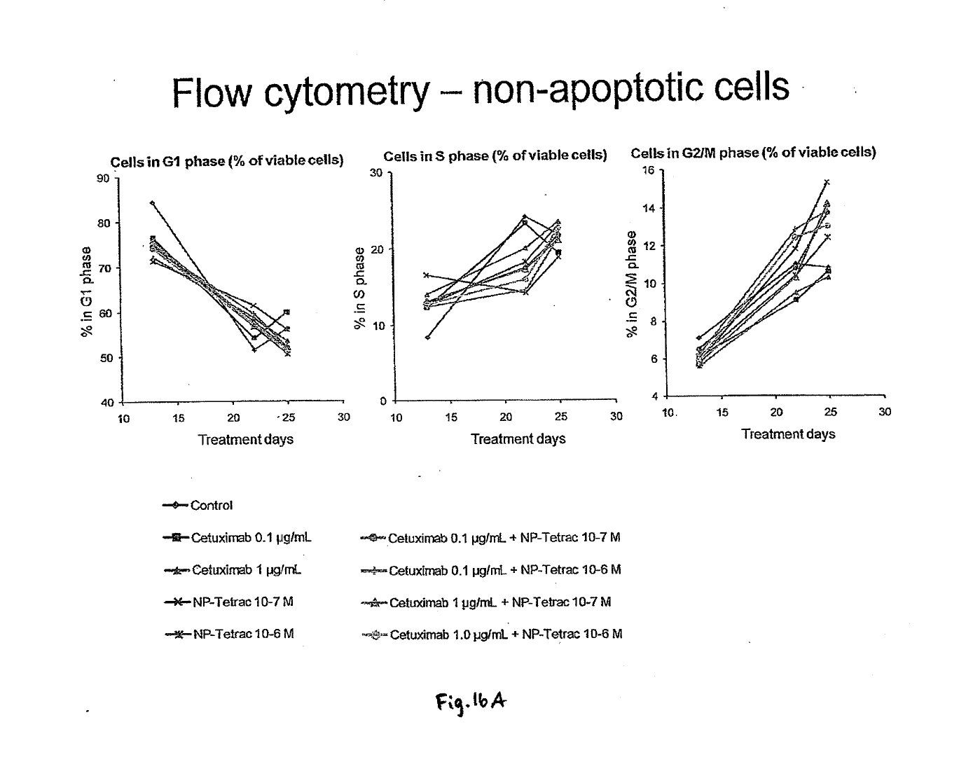

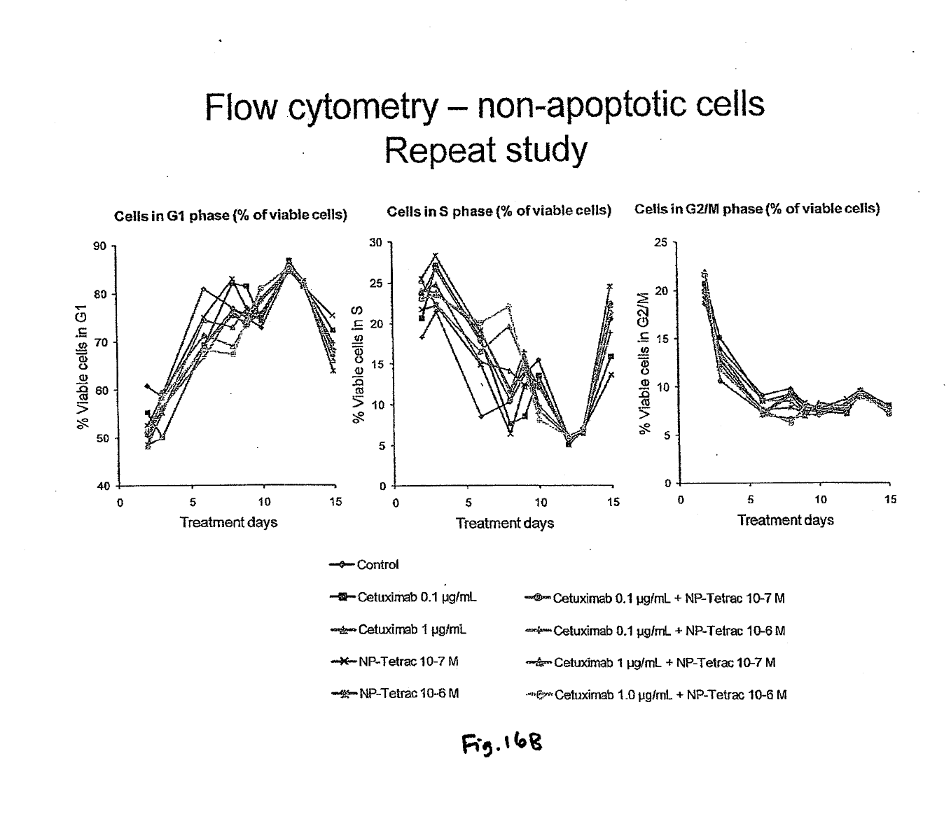

FIGS. 15A-C show the results of flow cytometry experiments on apoptosis following treatment with cetuximab, NP-Tetrac, or a combination. Cells in S phase were most sensitive to apoptosis. FIGS. 16A-B shows the results of flow cytometry experiments on non-apoptotic cells following treatment with cetuximab, NP-Tetrac, or a combination thereof.

These results show that the combined treatment with NP-Tetrac and cetuximab had a larger anti-proliferative effect on K-ras mutant cancer cells than cetuximab alone. A model assuming that both drugs act by separate pathways on success of replication adequately described the total cell counts from all treatments simultaneously.

The parameter estimates used herein are as follows:
Cetuximab: Iₘₐₓ 0.011, IC₅₀ 1.72 μg/mL
NP-Tetrac: Iₘₐₓ 0.053, IC₅₀ 0.104×10⁻⁶ M

The fraction of apoptotic cells was increased by up to a factor of 5 with the combination treatment versus treatment with cetuximab alone.

### CONCLUSION

Accordingly, based on the results presented herein, combined treatment with NP-Tetrac and cetuximab has a larger anti-proliferative effect on cancer cells than treatment with cetuximab alone. Moreover, NP-Tetrac induces apoptosis in cetuximab-resistant K-ras mutant colon cancer cells.

In combination with PD modeling, the use of the perfusion bellows cell culture system allows one to study the dose-response relationship of anti-neoplastic agents over a wide concentration range in vitro, and can support translation from in vitro to animal models and human clinical trials.

The addition of a cytotoxic drug after pre-treatment with NP-Tetrac and cetuximab may also be promising in the treatment of cancer.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An in vitro cell culture system comprising:
a) a compressible vessel comprising cell culture medium, a plurality of polymer flakes, and one or more porous membranes, wherein said polymer flakes have one or more cancer cells adhered thereto;
b) a media vessel used to supply the cell culture medium to the compressible vessel, wherein the media vessel is attached to the compressible vessel via a first connection line having at least one access point where one or more anti-cancer drugs or chemotherapeutic agents can be added to the cell culture medium;
c) a waste vessel used to remove waste products from the compressible vessel, wherein the waste vessel is attached to the compressible vessel via a second connection line having at least one access point where samples of the cell culture medium from the compressible vessel can be removed for analysis; and
d) a hollow fiber tube that connects the media vessel and the compressible vessel.

2. The in vitro cell culture system of claim 1, wherein the hollow fiber tube provides an environment for endothelial cells to grow.

3. The in vitro cell culture system of claim 2, wherein the hollow fiber tube allows the study of the effect of anti-cancer drug or chemotherapeutic agent-induced anti-angiogenesis.

4. The in vitro cell culture system of claim 2, wherein the hollow fiber tube allows the study of the effect of angiogenesis induced by cancer cells grown in the compressible vessel in the control set and the study of anti-cancer drug or chemotherapeutic agent-induced anti-angiogenesis.

5. The in vitro cell culture system of claim 1, wherein the compressible vessel is a compressible (bellows) bottle.

6. The in vitro cell culture system of claim 1, wherein the one or more cancer cells spontaneously adhere to the polymer flakes in the compressible vessel and proliferate.

7. The in vitro cell culture system of claim 1, wherein when the compressible vessel is compressed, the level of cell culture medium within the compressible vessel changes.

8. The in vitro cell culture system of claim 7, wherein, when the compressible vessel is compressed, the cells adhered to the polymer flakes are alternatively submerged in the cell culture medium and exposed to 5% CO₂/95% air.

9. The in vitro cell culture system of claim 8, wherein there is a dynamic interface between air and medium on the cells adhered to the polymer flakes that maximizes nutrient uptake and oxygen transfer by the cells.

10. The in vitro cell culture system of claim 1, wherein the system provides a low shear, high aeration, and foam-free cell culture environment.

11. The in vitro cell culture system of claim 1, wherein the polymer flakes are treated to enable seeding and the harvesting of cells and secreted proteins.

12. The in vitro cell culture system of claim 1, wherein between 10-15×10⁶ and 30-50×10⁶ cells are attached to the polymer flakes.

13. A method of using the in vitro cell culture system of claim 1, the method comprising the steps of:
a) seeding 5×10⁷ cell onto polymer flakes in perfusion and non-perfusion bellows bottles;
b) incubating the cells overnight;
c) harvesting the flakes;
d) placing the harvested flakes in the compressible vessel and allowing the system to run for at least 2 days;
e) exposing the cultured cells to serum-containing medium;
f) adding an amount of one or more anti-cancer drugs or chemotherapeutic agents via the access point in the first connection line for a period of up to 60 days.

14. The method of claim 13, wherein the compressible vessel is alternatively compressed and released during the period of up to 60 days.

15. The method of claim 14, further comprising the step of harvesting cell culture medium; soluble metabolites for cell culture; metabolites or anti-cancer drugs or chemotherapeutic agents; or any combination thereof from the access point in the second connection line for analysis.

16. The method of claim 15, wherein the analysis involves one or more pharmacodynamic studies selected from the group consisting of flow cytometry for cell cycle arrest or subpopulations of cells; microarray analysis for gene profiles; RT-PCR for gene expression; Western blot for anti-apoptotic or pro-apoptotic proteins; and direct cell counts.

17. The method of claim 14, further comprising the step of harvesting cells from the polymer flakes for analysis.

18. The method of claim 17, wherein the analysis of the harvested cells is selected from the group consisting of measurements of cell cycling, measurement of apoptosis, analysis of gene expression, anti-proliferation from direct cell number counts, and subpopulations of drug sensitive or resistant cells.

19. The method of claim 13, further comprising using mathematical modeling to consider the entire time course of cell counts in response to multiple concentrations of the one or more anti-cancer drugs or chemotherapeutic agents.

20. The method of claim 13, wherein the amount of the one or more anti-cancer drugs or chemotherapeutic agents is constant.

21. The method of claim 13, wherein the amount of the one or more anti-cancer drugs or chemotherapeutic agents is added by infusion.

22. The method of claim 13, wherein the amount of the one or more anti-cancer drugs or chemotherapeutic agents is added periodically.

23. The method of claim 13, wherein the amount of the one or more anti-cancer drugs or chemotherapeutic agents is varied by adjusting the flow rather of the cell culture medium, the dosing schedule, or both.

24. The method of claim 23, wherein the one or more anti-cancer drugs or chemotherapeutic agents is administered in multiple short-term infusions, in intermittent infusions, in brief injections, or in any combination thereof.
